(19) United States
(12) Patent Application Publication
(10) Pub. No.: US 2014/0047570 A1
(43) Pub. Date: Feb. 13, 2014

(54) ANIMAL MODEL OF HUMAN CANCER AND METHODS OF USE

(71) Applicant: Soner Altiok, Tampa, FL (US)

(72) Inventor: Soner Altiok, Tampa, FL (US)

(73) Assignee: H. LEE MOFFITT CANCER CENTER AND RESEARCH INSTITUTE, INC., Tampa, FL (US)

(21) Appl. No.: 14/113,014

(22) PCT Filed: Apr. 19, 2012
(86) PCT No.: PCT/US12/34290

§ 371 Date: Oct. 21, 2013

Related U.S. Application Data
(60) Provisional application No. 61/477,101, filed on Apr. 19, 2011.

(51) Int. Cl. A61K 49/00

(52) U.S. Cl. CPC A61K 49/0008
USPC 800/3; 800/10; 435/29; 435/6.11

(57) ABSTRACT

The subject invention pertains to a non-human animal model of human cancer, methods of producing a non-human animal model of human cancer, methods of using a non-human animal model to propagate human cancer cells, methods of using a non-human animal model to study cancer, methods of using a non-human animal model to screen potential treatments for a subject's cancer, methods of using a non-human animal model for treating cancer in a subject (providing personalized therapy), methods of using a non-human animal model for identifying a biomarker of cancer treatment; and methods of using a non-human animal model for selecting cancer patients for a clinical trial.

### CROSS-REFERENCE TO RELATED APPLICATION

The present application claims the benefit of U.S. Provisional Application Ser. No. 61/477,101, filed Apr. 19, 2011, which is hereby incorporated by reference herein in its entirety, including any figures, tables, or drawings.

### BACKGROUND OF THE INVENTION

The advances in molecular biology have afforded an increasingly sophisticated understanding of the molecular pathogenesis of cancer, which has led to the characterization of biologically important signaling pathways in cancer, the elements of which have focused drug development efforts toward novel, targeted therapies. The clinical development of targeted therapeutics, however, continues to be a largely empirical process. Recent studies show that only five percent of cancer drugs under development are actually approved. The majority of new cancer treatments fail due to lack of efficacy in patients, indicating that the current cell-based pre-clinical methods of testing cancer drug efficacy have limited accuracy and that traditional drug development paradigms may not be ideally suited to realize the full clinical potential of these new agents. Such failure in drug development comes at a large financial cost per drug, not to mention the human toll exacted in the process.

High clinical development costs coupled with declining drug discovery success rates have led pharmaceutical companies to re-evaluate their drug development process in order to reduce attrition rates and remain competitive. Given their potential for effective prioritization of drug development resources, biomarker studies are expected to change the way in which pharmaceutical companies determine the economic viability of their drug discovery process. The use of biomarkers would not only aid the discovery of promising products, it will also create an enhanced understanding of the clinical development process and help to facilitate the shift towards “personalized medicine”.

Traditional approaches to the preclinical investigation of novel cancer therapies rely mainly on the use of established human cancer cell lines (also referred to as tumor cell lines). These cell lines are maintained in vitro in serum based growth media, and their responses to experimental cancer therapeutic agents is assessed by studying growth and apoptosis in vitro or in vivo. Examples of cancer cell lines include, but are not limited to, bladder cancer cell lines (RT112, SW780), brain cancer cell lines (D54, SF-295, SK-N-AS, U87 MG), breast cancer cell lines (BT474, JIMT-1, MCF-7, MDA-MB-231, MX-1, ZR-75-1), colon cancer cell lines (COLO 205, DLD-1, HCT 116), HCT-15, HT-29, LoVo, LS-174T, SW-620, SW-480), fibrosarcoma cell line (HT-1080), gastric cancer cell lines (MKN-45, NCI-NC87), SNU-5), head and neck cancer cell lines (FADu, HONE-T-1), hepatocellular cancer cell line (SNU-398), Leukemia/lymphoma cell lines (Daudi, DoHH-2, Granta 519, HL-60, K-562, MOLT-4, MV4-11, Namalwa, Raji B, Ramos, REC-1, RL, WSU-DLCL2), liver cancer cell line (Hep3B), lung cancer cell lines (A-427, A549, Calu-6, NC1-H125, H1975, NC1-H23, MV-522, NC1-H1299, NC1-H345, NC1-H460, NC1-H520, NC1-H522, NC1-H69, SK-MES-1), melanoma cell lines (A2058, A375, Malme 3M, SK-MEL-5), multiple myeloma cell lines (H929, OPM-2, RPMI 8226), osteosarcoma cell line (SJSA-1), ovarian cancer cell line (A2780, IGR-OV1, OV-CAR-3, SK-OV-3), pancreatic cancer cell lines (BxPc-3, Caan-1, MIA PaCa-2, PANC-1), prostate cancer cell lines (22Rv.1, Du 145, PC3), renal cancer cell lines (786-0, A498, Caki-1, Caki-2, G-401, G-402), thyroid cancer cell lines (8505C, FTC-238), and vulvar/epitheloid cancer cell line (A-431).

Prolonged culture of human cancer cells in serum and on tissue culture plastic results in cell lines that may not be representative of the parent tumor. Such differences are of concern in the study of basic cancer biology, and are fundamental to our approach in drug discovery and development. In particular, culture selection in cell lines may disturb the in vitro relationship between the cancer stem cell and its progeny, and removes the contribution of tumor-stromal interactions, which are important to the three dimensional biology of solid tumors in vivo. In order for novel therapeutic and diagnostic strategies to be investigated with greater accuracy, new preclinical strategies are needed to assess anti-cancer therapies.

Primary human xenograft models of cancer typically involve the acquisition of tumor tissue from the operating room at the time of surgery and implantation directly into immunodeficient mice. The utility of primary human cancer xenografts as a platform to study cancer biology and to develop novel therapeutic and diagnostic approaches to cancer has been demonstrated (Rubio-Viqueira et al., Mol Cancer Ther., 2007:6:515-23; Rubio-Viqueira et al., Clin Cancer Res., 2006, 12:4652-61; Hidalgo et al., Mol Cancer Ther., 2006, 5:1895-903). Studies have shown that these tumors maintain the main features of the originating cancer; hence, it is believed that their use in preclinical studies reproduces more accurately the clinical scenario compared with studies done with cell lines. This technique, however, is rarely used in drug development and biomarker discovery efforts in the pharmaceutical industry mainly due to limited availability of low passage xenograft models with reliable clinical information. Furthermore, utilization of patient-derived xenograft mouse models has also been hindered by high cost and ethical issues related to the consumption of large numbers of mice in conventional drug treatment studies.

### BRIEF SUMMARY OF THE INVENTION

This invention involves establishment of a liver xenograft animal model bearing one or more primary human cancer cells. The inventor has demonstrated that fresh tumor tissue obtained by core biopsy, fine needle aspiration biopsy (FNAB) or prepared by mechanical mincing of tumor obtained by surgery can safely be implanted in or on the liver of an immunodeficient animal (such as a mouse) for propagation, drug testing, biomarker discovery/validation and personalized cancer therapy purposes. The inventor has showed that tumor growth in the liver can be monitored by in vivo imaging techniques such as ultrasound (US) and anatomical and diffusion magnetic resonance imaging (MRI) methods. The inventor's data revealed that imaging techniques such as diffusion MRI can detect tumor response to the drug treatment at the early stage of treatment in liver. The inventor's data indicate that tumor take rate in the liver is faster and higher then conventional subcutaneous (SC) implantation.

The liver is the second most commonly involved organ by metastatic cancer, after the lymph nodes, and may be the site of metastasis from virtually any primary malignant neoplasm. The liver provides a fertile ground for metastases, not only due to its rich, dual blood supply but also because of growth factors that promote cell growth. Liver involvement of metastatic tumor and the duration of survival appear to be inversely related. Therefore, the animal liver microenvironment is more representative of human tumor, especially in the metastatic setting, and that the liver xenograft model is more representative of clinical scenarios than other xenograft cancer models including heterotopic SC models.

The animal model of the invention can be used for drug development and drug testing; biomarker discovery and validation in the tumor, surrogate tissue and serum/plasma; personalized therapy whereby tumor cells from individual patients can be implanted in or on the animal liver and tested for the most effective drug/drug combinations in a short period of time; and fast and cost effective propagation of human tumor for subsequent proteomic, genomic and analytical analysis.

One aspect of the invention concerns an animal model comprising a non-human animal having one or more primary human cancer cells (not cells of a cancer cell line) implanted in or on the liver of the animal. The one or more human cancer cells may be obtained directly from a human tumor (e.g., biopsy material), for example, or from a primary culture. Preferably, a plurality of primary human cancer cells are implanted in or on the liver of the animal. Preferably, the implanted cancer cells exhibit a state of growth (propagation) in or on the liver. The implanted cancer cells may originate from a primary tumor or from a metastasized tumor. The implanted cancer cells may be orthotopic (originating from the same anatomic location as the site of implantation, the liver) or heterotopic (obtained from an anatomic site other than liver).

In some embodiments, the implanted cells originated outside the donor liver and metastasized to the donor liver. Thus, for example, the one or more cancer cells to be implanted may be metastatic human cancer cells that originated outside the human's liver and metastasized to the human's liver.

In some embodiments, the cancer cells are implanted within the liver. An incision of the skin and underlying fascia is made in the animal. The liver is exposed and optionally removed through the incision of the skin. An incision is made in the liver and tumor tissue is placed in the liver via the liver incision. The incision in the liver is then sealed to avoid internal bleeding following the procedure. For example, a surgical sealant and/or hemostatic agent may be applied to the incision of the liver. Preferably, a non-invasive hemostatic patch is placed over the incision of the liver.

The implanted cells may bear a detectable label (e.g., a bioluminescent label such as luciferase). The implanted cells may carry a heterologous nucleic acid. The nucleic acid may encode, for example, a detectable label.

The animal may be any non-human animal having a liver on or in which the primary cancer cells may be implanted. For example, the animal may be a mouse, rat, hamster, or other rodent; rabbit, pig, guinea pig, or dog, among others. Preferably, the animal is a mouse. In preferred embodiments, the subject from which the one or more primary cancer cells are obtained (the subject having the cancer) is human. However, in some embodiments of the invention (e.g., for veterinary oncology applications), the subject is a non-human animal.

Optionally, the animal is immunodeficient (a condition under which: a portion or some portions of cell components constituting an immune system are defective or dysfunctional, so that a normal immune mechanism is damaged). The immunodeficiency may be congenital or acquired. In some embodiments, the animal is immune deficient such that immunocompetent cells or factors involved in immune response are partially or entirely defective. For example, it may be preferable that the immune deficient animal is an animal whose T-cell and/or B-cell-dependent immune response capability is defective. Further, it may be preferable that the immune-deficient animal is an animal whose natural-killer-cell (NK cell) dependent immune response capability is defective (or the immune response capability is suppressed). When many immunocompetent cells or many factors involved in immune response are defective, it may be possible to suppress the immune response dependent on the cells or the factors, so that it may be possible to depress rejection at the time of transplantation of the one or more primary cancer cells implanted in or on the animal's liver.

Therefore, non-limiting examples of the immune deficient animal include: a nude animal whose T-cell-dependent immune response capability is defective since the nude animal has no thymus; a scid animal whose B-cell-dependent immune response capability is defective as well as the immune response capability of the nude animal; and an animal whose NK-cell-dependent immune response capability is defective as well as the immune response capability of the scid animal.

Optionally, the animal is genetically engineered. Genes may be over-expressed or under-expressed (e.g., knocked out) in the animal, such as beta-2 microglobulin (B2m), forkhead box N1 (Foxn1), interleukin 2 receptor (Il2rg), perforin 1 (Prf1), protein kinase (Prkdc), and recombination activating gene (Rag1). Animals with various genetic backgrounds are known in the art and may be utilized to produce an animal model of the invention, such as BALB substrains (e.g., CByJ.Cg-Foxn1^{nu}/J, or CBySmn.CB17-Prkdc^{scid}/J), C57BL/6 J (e.g., B6;129S7-Rag1^{tm1Mom}/J, B6.129S7-Rag1^{tm1Mom}/J, or B6.CB17-Prkdc^{scid}/SzJ), NOD/LtSzJ (e.g., NOD.129S7(B6)-Rag1^{tm1Mom}/J NOD.Cg-Rag1^{tm1Mom}Prf1^{tm1Sdz}/Sz, NOD.CB17-Prkdc^{scid}/SzJ, NOD.Cg-Prkdc^{scid}B2m^{tm1Unc}/J, or NOD.Cg-Prkdc^{scid}Il2rg^{tm1Wjlc}/SzJ), and NU/J (The Jackson Laboratory, Bar Harbor, Me.). In some embodiments, the animal is a mouse selected from among CB 17-Prkdc(Scid) (CB 17-scid) mice, NOD-scid mice, or mice bearing a targeted mutation in the IL-2 receptor common gamma chain (IL2rgamma(null)).

Another aspect of the invention concerns a method of producing an animal model of the invention, comprising implanting one or more primary human cancer cells in or on the liver of a non-human animal.

Another aspect of the invention concerns a method of propagating human cancer cells, comprising implanting one or more primary human cancer cells in or on the liver of a non-human animal; and allowing the implanted cells to propagate. In some embodiments, the one or more human cancer cells are obtained directly from a human tumor (e.g., biopsy material), or the one or more human cancer cells are cells of a primary culture. In some embodiments, the method further comprises harvesting the propagated cancer cells from the animal after the cells have been allowed to propagate in the animal. In some embodiments, the method further comprises evaluating at least one parameter of the harvested cancer cells (e.g., proteomic analysis, genomic analysis, analytical analysis). Optionally, cancer cells harvested from the animal may be placed in storage. In some embodiments, the method further comprises culturing (expanding) the harvested cancer cells and, optionally, storing the harvested cells. Optionally, harvested cancer cells may be cultured and/or stored and one or more of the cultured and/or stored cells may be implanted in or on the liver of one or more other non-human animals (this process may be carried out repeatedly—in series, in parallel, or both).

Another aspect of the invention concerns a method of evaluating human cancer cell growth, comprising providing an animal model of the invention, and evaluating the growth of the one or more primary human cancer cells in or on the liver of the animal. Evaluation of cancer cell growth following implantation can be carried out ex vivo or in vivo. Preferably, evaluation of cancer cell growth is carried out in the animal in vivo. For example, evaluation of cancer cell growth may be carried out in vivo with an imaging modality selected from among one or more of bioluminescent imaging (e.g., luciferase), ultrasound imaging, fluorescence molecular tomography (FMT), and magnetic resonance imaging (e.g., anatomical MRI, diffusion MRI, MRI spectroscopy, dynamic contrast enhanced (DCE) MRI). In some embodiments, a biologically active agent is administered to the animal before, during, and/or after implantation of the one or more primary human cancer cells and the response of the cancer cells to the biologically active agent is evaluated ex vivo or in vivo. In some embodiments, a cancer treatment is administered to the animal before, during, and/or after implantation of the one or more primary human cancer cells and the response of the cancer cells to the treatment is evaluated ex vivo or in vivo. For example, cancer cell growth may be evaluated ex vivo or in vivo in response to a cancer treatment or to a biologically active agent. Optionally, a combination of biologically active agents is administered and its effect is evaluated. In some embodiments, the biologically active agent is a chemotherapeutic agent or other anti-cancer agent. However, the biologically active agent may be a non-anti-cancer agent. Optionally, the method of evaluating human cancer cell growth further comprises recording the sensitivity/resistance of the one or more human cancer cells to the anti-cancer agent in a computer readable medium.

Another aspect of the invention includes a method of studying human cancer, comprising providing the animal model of the invention, and evaluating at least one parameter of the one or more primary human cancer cells and/or the animal model. In some embodiments, the parameter comprises the presence or absence of a biomarker. In some embodiments, the biomarker comprises one or more tumor markers. In some embodiments, the biomarker is a gene expression signature and the method further comprises recording the gene expression signature (e.g., an expression level) in a computer readable medium.

Another aspect of the invention concerns a method for screening potential treatments for a cancer in a subject, comprising implanting one or more primary human cancer cells from the subject in or on the liver of a non-human animal; administering a candidate treatment to the animal before, during, or after said implanting; and evaluating at least one parameter of the one or more implanted primary human cancer cells and/or the animal that is associated with cancer treatment efficacy or lack of efficacy. The candidate treatment may be, for example, a chemotherapeutic treatment or other anti-cancer treatment, such as an immunologic treatment, a radiation treatment, or any combination of two or more anti-cancer treatments. The parameter(s) evaluated may be parameters of the cancer cells and/or the animal that provide information as to whether the candidate treatment is effective in treating the cancer. For example, the at least one parameter may comprise cancer cell growth rate or tumor size. In some embodiments, the evaluation comprises imaging at least a portion of the animal to determine the response of the one or more human cancer cells to the candidate treatment. Imaging can be carried out, for example, with an imaging modality selected from among one or more of bioluminescent imaging (e.g., luciferase), ultrasound imaging, fluorescence molecular tomography (FMT), and magnetic resonance imaging (e.g., anatomical MRI, diffusion MRI, MRI spectroscopy, dynamic contrast enhanced (DCE) MRI). In some embodiments, implanting comprises implanting one or more primary human cancer cells from the subject in or on the liver of a plurality of non-human animals, and the administration step comprises administering a candidate treatment to each animal before, during, or after implanting the one or more cancer cells. In some embodiments, a different candidate treatment is administered to each animal. In some embodiments, in order to obtain information concerning effective dose or optimum dose, a different dose of the same candidate treatment can be administered to each animal. In some embodiments of the screening method, the method further comprises selecting and administering the candidate treatment to the subject if the results of the evaluation are consistent with cancer treatment efficacy.

Another aspect of the invention includes a method for treating cancer in a subject, comprising selecting a candidate treatment from among a plurality of candidate treatments, and administering the selected treatment to the subject, wherein the selected candidate treatment has been determined to be effective in treating the cancer in a non-human animal model having one or more primary cancer cells from the cancer implanted in or on the liver of the animal.

Another aspect of the invention concerns a method for identifying a biomarker for cancer treatment, comprising providing a plurality of non-human animals having one or more primary human cancer cells from a plurality of humans implanted in or on the liver of the plurality of animals; administering a cancer treatment to the plurality of animals; identifying animals among the plurality of animals in which the cancer treatment is effective; and identifying a biomarker that is common to the identified animals and is associated with (correlates with) the treatment's effectiveness.

Another aspect of the invention concerns a method for identifying a biomarker for cancer treatment, comprising providing a plurality of non-human animals having one or more primary human cancer cells from a plurality of humans implanted in or on the liver of the plurality of animals; administering a cancer treatment to the plurality of animals; identifying animals among the plurality of animals in which the cancer treatment is ineffective; and identifying a biomarker that is common to the identified animals and is associated with (correlates with) the treatment's ineffectiveness.

Another aspect of the invention concerns a method for selecting cancer patients for a clinical trial, comprising providing a plurality of non-human animals having one or more primary human cancer cells from a plurality of humans implanted in or on the liver of the plurality of animals; administering a cancer treatment to the plurality of animals; identifying animals among the plurality of animals in which the cancer treatment is effective; identifying a biomarker that is common to the identified animals and is associated with (correlates with) the treatment's effectiveness; and including a patient in the clinical trial if the patient has the biomarker or excluding the patient from the clinical if the patient lacks the biomarker.

Another aspect of the invention pertains to a method for selecting cancer patients for a clinical trial, comprising providing a plurality of non-human animals having one or more primary human cancer cells from a plurality of humans implanted in or on the liver of the plurality of animals; administering a cancer treatment to the plurality of animals; identifying animals among the plurality of animals in which the cancer treatment is ineffective; identifying a biomarker that is common to the identified animals and is associated with (correlates with) the treatment's ineffectiveness; and including a patient in the clinical trial if the patient lacks the biomarker or excluding the patient from the clinical trial if the patient has the biomarker.

Another aspect of the invention is an animal model comprising a non-human animal having one or more primary cancer cells implanted in or on the liver of said animal, wherein the primary cancer cells are from a species different from that of the animal. Optionally, the species of the primary cancer cells implanted to the animal is human, as described above. However, the primary cancer cells may be obtained from a non-human animal species, in which case the animal model may be used for carrying out the aforementioned methods of the invention except that the area of study is veterinary (veterinary oncology) and the patient is the non-human animal (a veterinary patient) from which the primary cancer cells are obtained. In some embodiments, the implanted cells originated outside the donor liver and metastasized to the donor liver. Thus, for example, the one or more cancer cells to be implanted may be metastatic cancer cells that originated outside the donor's liver and metastasized to the donor's liver.

In some embodiments, the cancer cells are implanted within the liver. An incision of the skin and underlying fascia is made in the animal model. The liver is exposed and optionally removed through the incision of the skin. An incision is made in the liver and tumor tissue is placed in the liver via the liver incision. The incision in the liver is then sealed to avoid internal bleeding following the procedure. For example, a surgical sealant and/or hemostatic agent may be applied to the incision of the liver. Preferably, a non-invasive hemostatic patch is placed over the incision of the liver.

### BRIEF DESCRIPTION OF THE DRAWINGS

FIG. 1 shows implantation of human cancer cells and tissue in the mouse liver. The purpose of these experiments was to determine the feasibility of the intrahepatic implantation of human tumor tissue and cells to create liver xenograft model of human cancer for drug testing, biomarker discovery/validation and personalized therapy of cancer patients. To visualize tumor growth in liver in this study the inventor used human cancer cells stably transfected with a luciferase expression vector. Cells were first injected subcutaneously (SC) into immunodeficient mice and once the SC tumor became 1 cm in diameter core biopsy and fine needle aspiration biopsy (FNAB) samples were collected, which were subsequently implanted into liver by laparotomy or direct injection. Tumor growth in SC and liver was measured by In Vivo Imaging System (IVIS) from Xenogen Corporation after injection of firefly Luciferin (150 mg/kg body weight) into the mice 10 minutes before imaging.

FIGS. 2A-C show monitoring of in vivo tumor growth in the liver by ultrasound imaging. In vivo tumor (T) growth in the liver (L) was monitored by ultrasound (US) imaging (FIG. 2A). Compared to images taken on day 3 (FIG. 2B), day 10 images (FIG. 2C) demonstrate an increase in the tumor size (right panel), demonstrating non-invasive in vivo imaging can be used for rapid and accurate measurement of tumor growth in liver.

FIG. 3 shows implantation of fresh human cancer tissue in the mouse liver. Fresh tumor tissue obtained from pancreatic (upper images) and lung (lower images) adenocarcinoma patients at the time of surgery were implanted in the liver of immunodeficient mice in the form of FNAB cell suspension, core biopsy or tissue fragments prepared by mechanical mincing. Seven day after the implantation, ultrasound (US) images were obtained to evaluate tumor growth. T: Tumor.

FIGS. 4A-D show MRI analysis to assess tumor response to drug treatment in vivo. To evaluate tumor response to treatment in vivo in addition to US imaging technologies such as MRI that utilizes anatomical MRI, diffusion MRI, spectroscopy and Dynamic Contrast Enhanced (DCE) MRI can be used. Of these, diffusion, spectroscopy and DCE show great promise in determining early response to drug treatment, even before changes in tumor volume are observed. Anatomical and diffusion MRI techniques were used on the same liver tumor of human pancreatic cancer before (FIG. 4A) and 24 hours after (FIG. 4B) the initiation of gemcitabine (intraperitoneally 1 mg/kg/d) treatment. Diffusion MRI showed a right shift in tumor water diffusion beginning by 24 hours after therapy demonstrating that in vivo imaging allows evaluation of tumor growth in the liver and early response to drug treatment (FIG. 4C). Laparoscopy or laparotomy can be used to assess tumor growth and to harvest tumor cells in liver (FIG. 4D).

FIGS. 5A-B show imaging of a selective non-peptide small molecule integrin αvβ3 antagonist (IntegriSense™ 750 targeted fluorescence imaging agent, PerkinElmer, Inc., Boston, Mass.) using a fluorescence molecular tomography (FMT) 2500 imaging system. FIG. 5A shows in vivo FMT imaging of αvβ3 expression of a tumor in the liver. FIG. 5B shows magnetic resonance imaging (MRI) in a patient-derived tumor implanted in the same mouse liver, which corroborated tumor location.

FIGS. 6A-B show histological correlation of tumors implanted in the mouse liver (FIG. 6A) and original patient tumors (FIG. 6B).

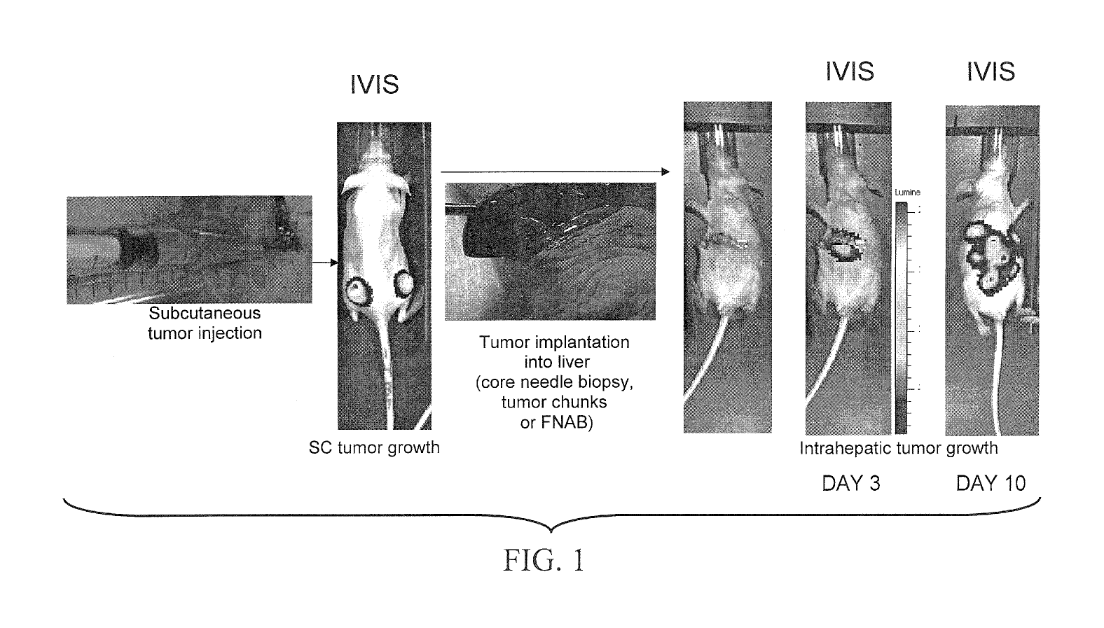

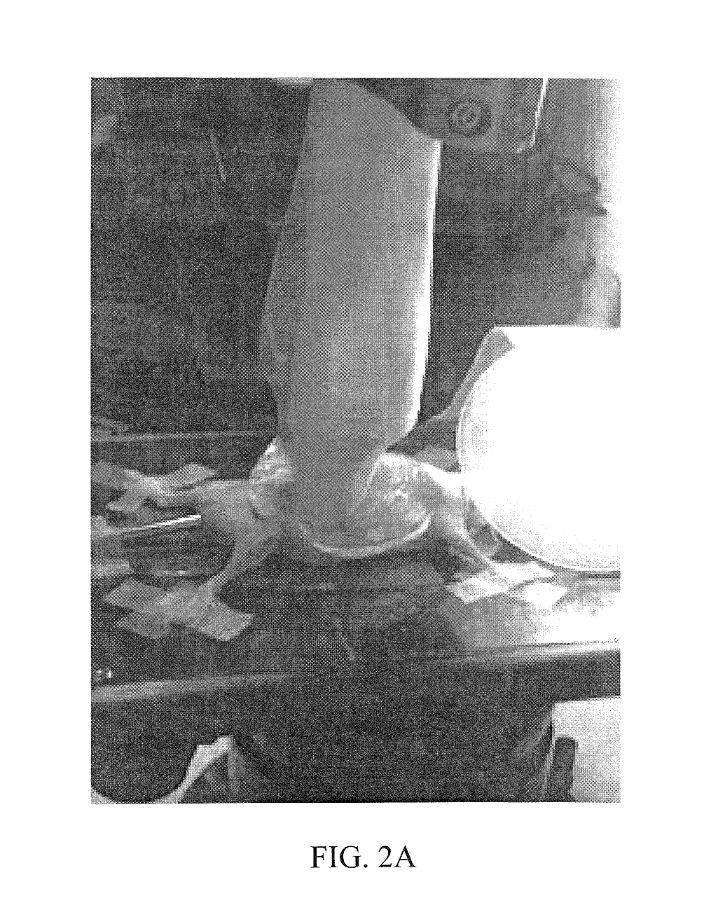

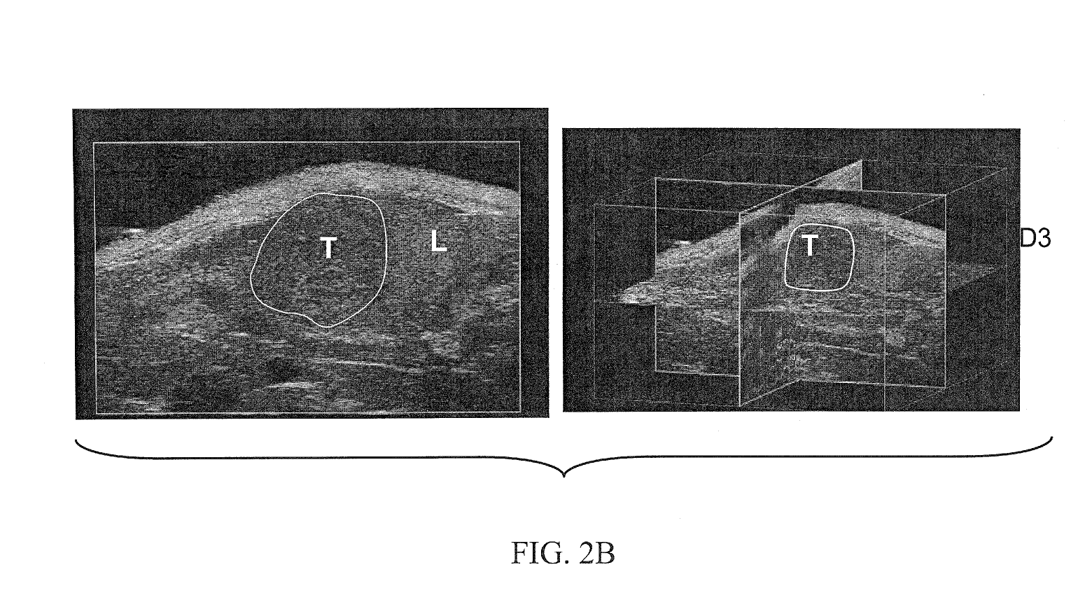

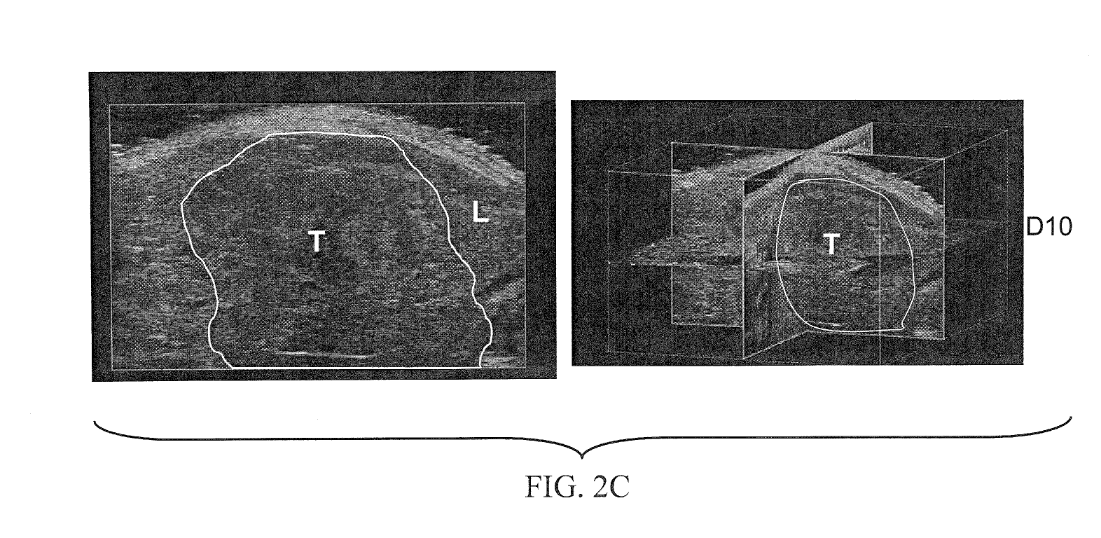

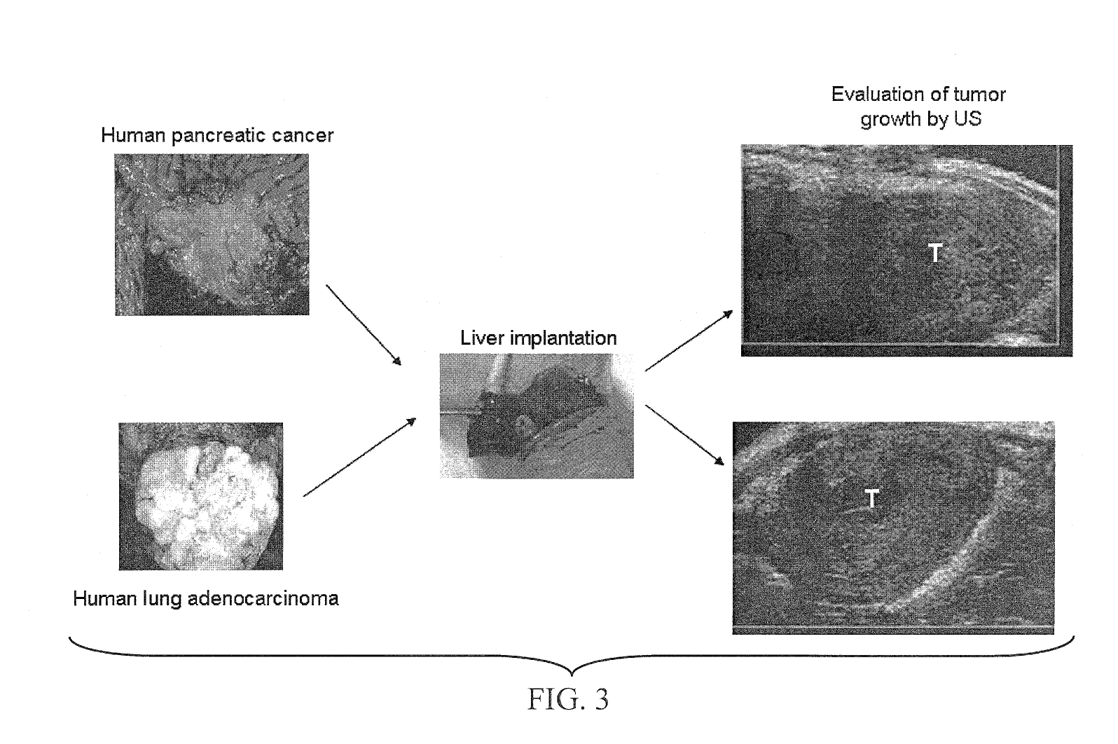

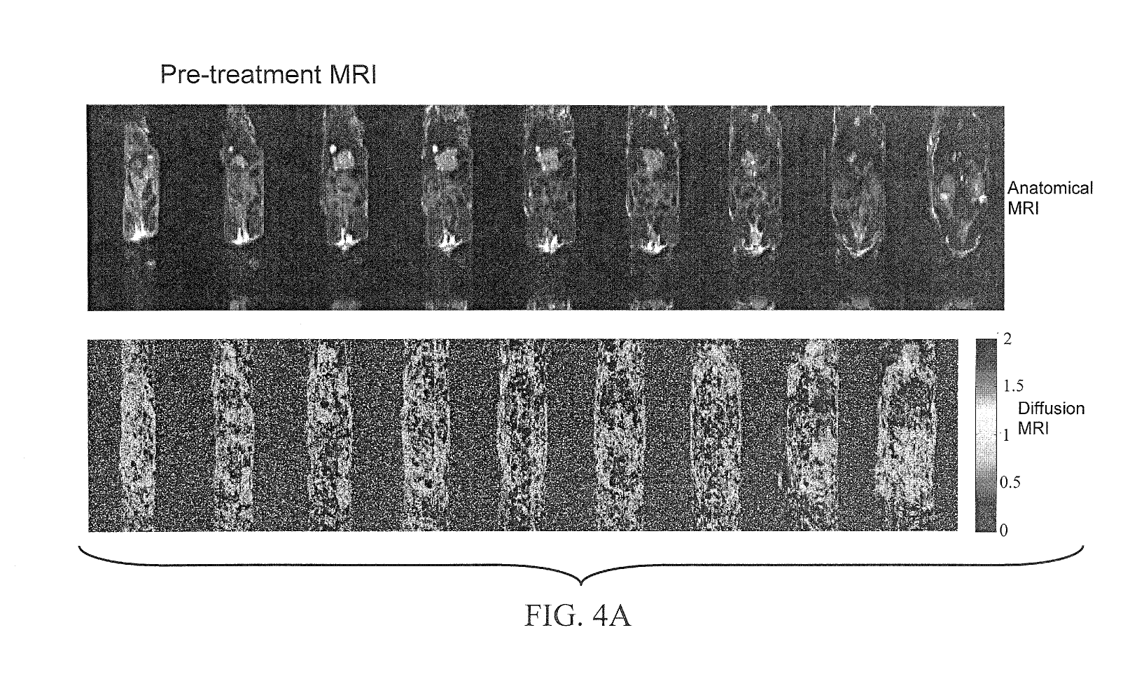

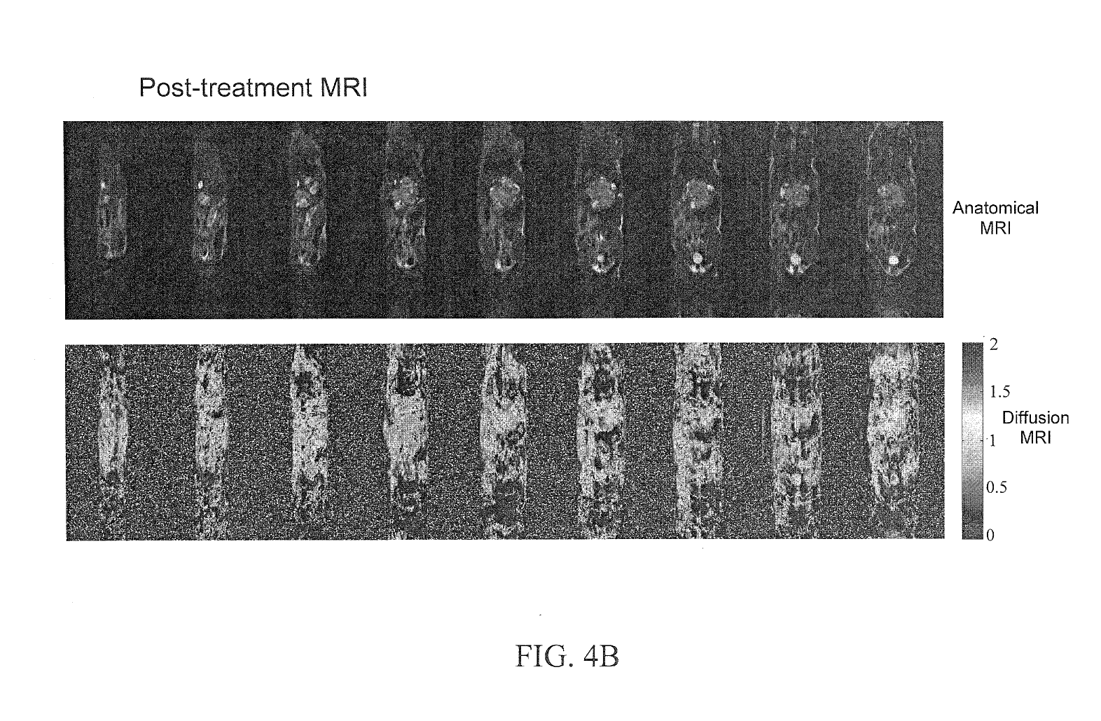

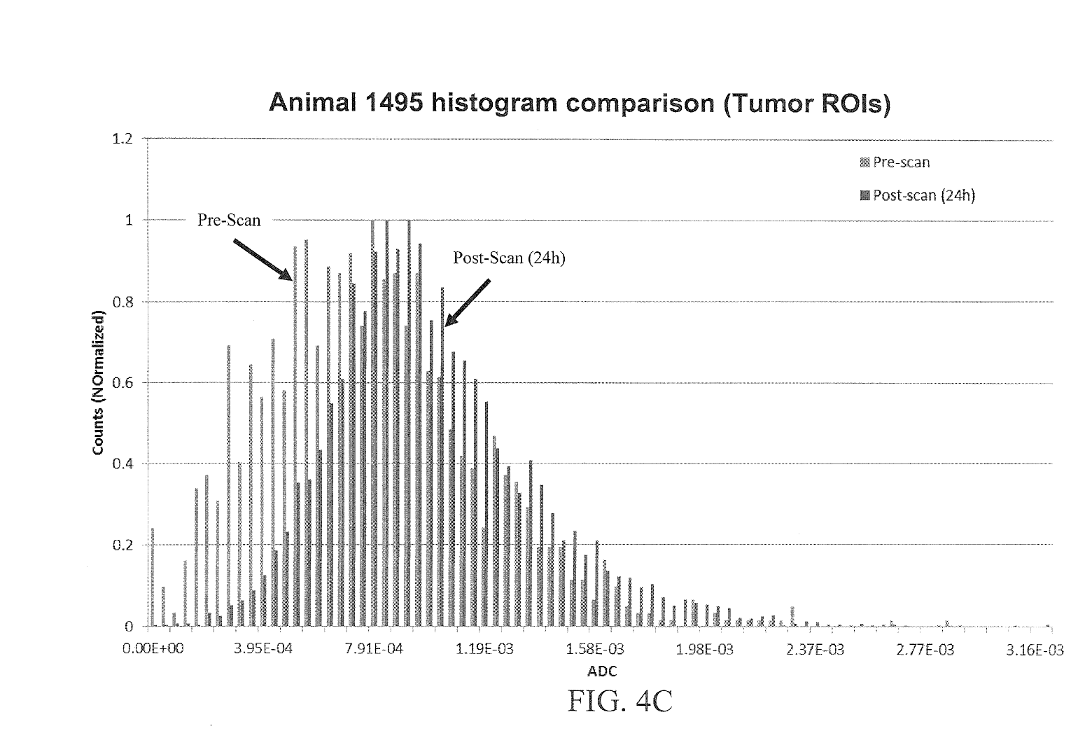

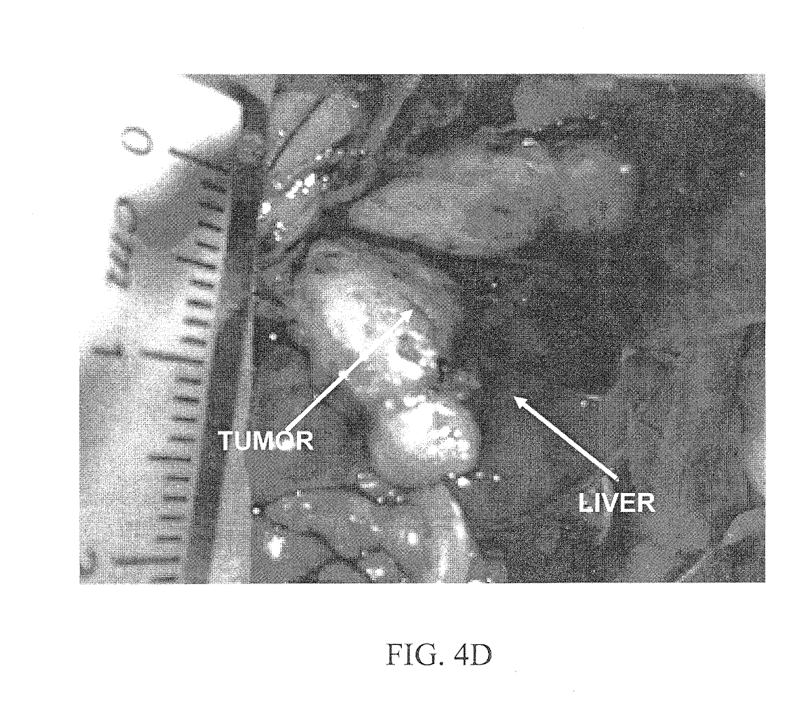

### DETAILED DISCLOSURE OF THE INVENTION

Liver is rich in nutrition and blood flow, and can provide a suitable environment for tumor growth. Therefore, the liver is the second most commonly involved organ by metastatic cancer and may be the site of metastasis from virtually any primary malignant neoplasm. Furthermore, liver involvement of metastatic tumor and the duration of survival appear to be inversely related. The invention provides an animal model that carries primary human cancer cells implanted in or on the liver. This model is more representative of clinical human cancer for the study drug effects, to identify and validate markers that can be used for diagnosis and prognosis of cancer, and prediction of drug treatment in the tumor. This approach can also be used for personalized tumor treatment in individual patients by selecting the most effective drug and drug combinations.

### I. Liver Xenograft Platform

One aspect of the invention concerns an animal model comprising a non-human animal having one or more primary human cancer cells (not cells of a cancer cell line) implanted in or on the liver of the animal. The one or more human cancer cells may be obtained directly from a human tumor (e.g., biopsy material), for example, or from a primary culture. Preferably, a plurality of primary human cancer cells are implanted in or on the liver of the animal. Preferably, the implanted cancer cells exhibit a state of growth (propagation) in or on the liver. The implanted cancer cells may originate from a primary tumor or from a metastasized tumor. The implanted cancer cells may be orthotopic (originating from the same anatomic location as the site of implantation, the liver) or heterotopic (obtained from an anatomic site other than liver).

In some embodiments, the implanted cells originated outside the donor liver and metastasized to the donor liver. Thus, for example, the one or more cancer cells to be implanted may be metastatic human cancer cells that originated outside the human's liver and metastasized to the human's liver.

The one or more primary cancer cells implanted in or on the liver of the non-human animal in accordance with the invention may be in isolated form, or may include other cells and/or materials (as a crude specimen), at the time of implantation. Optionally, the one or more primary cancer cells may be purified or undergo selection techniques (e.g., using flow cytometry) in order to implant only primary cancer cells or only subsets of primary cancer cells, such as cancer stem cells. In some embodiments, the cells are implanted as a tissue. Methods and markers commonly used to identify stein cells and to distinguish cell types are described in the scientific literature (e.g., Stem Cells: Scientific Progress and Future Research Directions, Appendix E1-E5, report prepared by the National Institutes of Health, June, 2001).

The number of primary cancer cells necessary for implantation and growth in or on the liver of the animal can be determined by those skilled in the art. In some embodiments, approximately 1,000-3,000 primary cancer cells are implanted. Any implantation technique effective in delivering the cells to the liver can be utilized. For example, the cells can be implanted in or on the liver in an open surgical manner (laparotomy) or through direct injection (e.g., intrahepatic injection).

An exemplified protocol for implantation is provided in Example 1. Briefly, donor tissue is removed and may be portioned, any necrotic tissue is preferably removed, and healthy tissue portions are re-suspended in fresh media. Tissue may be placed with media and constituted basement membrane (such as Matrigel™) or other cell culture substrate (preferably, in a 1:1 ratio). Optionally, tissue portions can be kept on ice prior to implantation. An incision of the skin and underlying fascia is made in the animal. The liver is exposed and optionally removed through the incision of the skin. Preferably, cells are implanted in the liver. Thus, an incision is made in the liver and tumor tissue is placed in the liver through the liver incision. The incision in the liver is then sealed to avoid internal bleeding following the procedure. For example, a surgical sealant and/or hemostatic agent may be applied to the incision of the liver. Preferably, a non-invasive hemostatic patch is placed over the incision of the liver. After confirming bleeding has stopped, the liver is returned to its position in the abdomen if it was removed (displaced) through the body incision for implantation. The incision in the skin and fascia is closed (e.g., with staples). Ketoprofen or other anti-inflammatory drugs may be administered to the animal, and saline may be administered for blood loss.

Optionally, the one or more primary cancer cells may be stored prior to implantation in the animal using methods known in the art (e.g., frozen) that would not be incompatible with the viability of the cells when implanted in or on the animal's liver. Suitable storage conditions will depend on the cancer type. Typically, cells of high grade, aggressive tumors survive better and longer than low grade tumor cells. If cancer cells are subsequently harvested from the animal model, the harvested cells may also be stored for a time. As described herein, harvested cells may be expanded (cultured) and implanted in multiple animals in series or parallel for further assessments, and this process may be repeated any number of times.

The implanted cells may bear a detectable label (e.g., a bioluminescent label such as luciferase). The implanted cells may carry a heterologous nucleic acid. The nucleic acid may encode, for example, a detectable label. Many detectable labels are known in the art and may be utilized with the invention. Depending upon the type, the label may be imaged using an imaging instrument.

The animal may be any non-human animal having a liver on or in which the primary cancer cells may be implanted. For example, the animal may be a mouse, rat, hamster, or other rodent; rabbit, pig, guinea pig, or dog, among others.

Optionally, the animal is immunodeficient (a condition under which: a portion or some portions of cell components constituting an immune system are defective or dysfunctional, so that a normal immune mechanism is damaged). The immunodeficiency may be congenital or acquired. In some embodiments, the animal is immune deficient such that immunocompetent cells or factors involved in immune response are partially or entirely defective. For example, it may be preferable that the immune deficient animal is an animal whose T-cell and/or B-cell-dependent immune response capability is defective. Further, it may be preferable that the immune-deficient animal is an animal whose natural-killer-cell (NK cell) dependent immune response capability is defective (or the immune response capability is suppressed). When many immunocompetent cells or many factors involved in immune response are defective, it may be possible to suppress the immune response dependent on the cells or the factors, so that it may be possible to depress rejection at the time of transplantation of the one or more primary cancer cells implanted in or on the animal's liver.

Therefore, non-limiting examples of the immune deficient animal include: a nude animal whose T-cell-dependent immune response capability is defective since the nude animal has no thymus; a scid animal whose B-cell-dependent immune response capability is defective as well as the immune response capability of the nude animal; and an animal whose NK-cell-dependent immune response capability is defective as well as the immune response capability of the scid animal.

Optionally, the animal is genetically engineered. Genes may be over-expressed or under-expressed (e.g., knocked out) in the animal, such as beta-2 microglobulin (B2m), forkhead box N1 (Foxn1), interleukin 2 receptor (Il2rg), perforin 1 (Prf1), protein kinase (Prkdc), and recombination activating gene (Rag1). Animals with various genetic backgrounds are known in the art and may be utilized to produce an animal model of the invention, such as BALB substrains (e.g., CByJ.Cg-Foxn1^{nu}/J, or CBySmn.CB17-Prkdc^{scid}/J), C57BL/6J (e.g., B6;129S7-Rag1^{tm1Mom}/J, B6.129S7-Rag1^{tm1Mom}/J, or B6.CB 17-Prkdc^{scid}/SzJ), NOD/LtSzJ (e.g., NOD.129S7(B6)-Rag1^{tm1Mom}/J, NOD.Cg-Rag1^{tm1Mom}Prf1^{tm1Sdz}/Sz, NOD.CB 17-Prkdc^{scid}/SzJ, NOD.Cg-Prkdc^{scid}B2m^{tm1Unc}/J, or NOD.Cg-Prkdc^{scid}Il2rg^{tm1Wjlc}/SzJ), and NU/J (The Jackson Laboratory, Bar Harbor, Me.). In some embodiments, the animal is a mouse selected from among CB 17-Prkdc(Scid) (CB 17-scid) mice, NOD-scid mice, or mice bearing a targeted mutation in the IL-2 receptor common gamma chain (IL2rgamma(null)).

Another aspect of the invention concerns a method of producing an animal model of the invention, comprising implanting one or more primary human cancer cells in or on the liver of a non-human animal.

Another aspect of the invention concerns a method of propagating human cancer cells, comprising implanting one or more primary human cancer cells in or on the liver of a non-human animal; and allowing the implanted cells to propagate. In some embodiments, the one or more human cancer cells are obtained directly from a human tumor (e.g., biopsy material), or the one or more human cancer cells are cells of a primary culture. In some embodiments, the method further comprises harvesting the propagated cells from the animal. In some embodiments, the method further comprises evaluating at least one parameter of the harvested cells (e.g., proteomic analysis, genomic analysis, analytical analysis).

Another aspect of the invention concerns a method of evaluating human cancer cell growth, comprising providing an animal model of the invention, and evaluating the growth of the one or more primary human cancer cells in or on the liver of the animal. Evaluation of cancer cell growth following implantation can be carried out ex vivo or in vivo. Preferably, evaluation of cancer cell growth is carried out in the animal in vivo. For example, evaluation of cancer cell growth may be carried out in vivo with an imaging modality selected from among one or more of bioluminescent imaging, (e.g., luciferase), ultrasound imaging, and magnetic resonance imaging (e.g., anatomical MRI, diffusion MRI, MRI spectroscopy, dynamic contrast enhanced (DCE) MRI). Depending on the modality utilized, labels and imagining agents may be re-administered to the cancer cells or the animal model periodically (e.g., by injection) as needed (e.g., for longitudinal studies). Optionally, pre-administration and pre-implantation images may be taken for comparison to subsequent images taken under the same or different conditions for evaluation of cancer cell growth (e.g., post-treatment). In some embodiments, a biologically active agent is administered to the animal before, during, and/or after implantation of the one or more primary human cancer cells and the response of the cancer cells to the biologically active agent is evaluated ex vivo or in vivo. In some embodiments, a cancer treatment is administered to the animal before, during, and/or after implantation of the one or more primary human cancer cells and the response of the cancer cells to the treatment is evaluated ex vivo or in vivo. For example, cancer cell growth may be evaluated ex vivo or in vivo in response to a cancer treatment or to a biologically active agent. Optionally, a combination of biologically active agents is administered and its effect is evaluated. In some embodiments, the biologically active agent is a chemotherapeutic agent or other anti-cancer agent. However, the biologically active agent may be a non-anti-cancer agent. Optionally, the method of evaluating human cancer cell growth further comprises recording the sensitivity/resistance of the one or more human cancer cells to the anti-cancer agent in a computer readable medium.

Another aspect of the invention includes a method of studying human cancer, comprising providing the animal model of the invention, and evaluating at least one parameter of the one or more primary human cancer cells and/or the animal model. In some embodiments, the evaluation includes gene expression profiling the cancer cells after implantation and/or the animal after implantation. In some embodiments, the parameter comprises the presence or absence of a biomarker (e.g., a single nucleotide polymorphism (SNP)). In some embodiments, the biomarker comprises one or more tumor markers. In some embodiments, the biomarker is a gene expression signature and the method further comprises recording the gene expression signature (e.g., an expression level) in a computer readable medium.

Another aspect of the invention is an animal model comprising a non-human animal having one or more primary cancer cells implanted in or on the liver of said animal, wherein the primary cancer cells are from a species different from that of the animal. Optionally, the species of the primary cancer cells implanted to the animal is human, as described above. However, the primary cancer cells may be obtained from a non-human animal species, in which case the animal model may be used for carrying out the aforementioned methods of the invention except that the area of study is veterinary and the patient is the non-human animal (a veterinary patient) from which the primary cancer cells are obtained. For example, the one or more primary cancer cells can be those of a domesticated farm animal or pet, or other non-human animal.

### II. Personalized Therapy; Drug Discovery and Development

Another aspect of the invention concerns a method for screening potential treatments for a cancer in a subject, comprising implanting one or more primary human cancer cells from the subject in or on the liver of a non-human animal; administering a candidate treatment to the animal before, during, or after said implanting; and evaluating at least one parameter of the one or more implanted primary human cancer cells and/or the animal that is associated with cancer treatment efficacy or lack of efficacy. The candidate treatment may be, for example, a chemotherapeutic treatment or other anti-cancer treatment, such as an immunologic treatment, a radiation treatment, or any combination of two or more anti-cancer treatments. The parameter(s) evaluated may be parameters of the cancer cells and/or the animal that provide information as to whether the candidate treatment is effective in treating the cancer. For example, the at least one parameter may comprise cancer cell growth rate or tumor size. In some embodiments, the evaluation comprises imaging at least a portion of the animal to determine the response of the one or more human cancer cells to the candidate treatment. In some embodiments, implanting comprises implanting one or more primary human cancer cells from the subject in or on the liver of a plurality of non-human animals, and the administration step comprises administering a candidate treatment to each animal before, during, or after implanting the one or more cancer cells. In some embodiments, a different candidate treatment is administered to each animal. In some embodiments, in order to obtain information concerning effective dose or optimum dose, a different dose of the same candidate treatment can be administered to each animal. In some embodiments of the screening method, the method further comprises selecting and administering the candidate treatment to the subject if the results of the evaluation are consistent with cancer treatment efficacy.

A highly sensitive and cost effective short term functional pharmacodynamic assay (MATEX) has been developed that simultaneously analyzes multiple signaling pathways in small human tumor tissue explants and fine needle aspiration biopsy samples in a quantitative manner with each tumor becoming its own control. This approach allows the screening of a large number of drugs to predict their in vivo efficacy before systemic treatment. This approach can be used with the animal models and methods of the invention to allow enrichment of “xenograft trials” by pre-selecting the most effective drugs for each patient tumor line to obtain meaningful data in a most cost effective way and by using the smallest number of animals, which would significantly decrease the cost of drug treatment studies. Previous and ongoing clinical studies have shown that tumor cells obtained by endoscopic/core or fine needle aspiration biopsies prior to initiation of therapy can be successfully assayed ex vivo to predict the in vivo pharmacodynamic effects of targeted drugs in cancer patients (Altiok et al., Int J. Oncol. 2010, 36:19-27). Thus, in some embodiments, the method further comprises obtaining a sample of cancer cells from the subject and assessing the therapeutic potential of a treatment (such as an anti-cancer agent) ex vivo, as a pre-screen, before screening potential treatments in the animal model of the invention. Methods assessing therapeutic potential include those described in U.S. Patent Publication No. 2009/0325202 (Altiok), which is incorporated herein by reference in its entirety. For example, the method may comprise obtaining a sample of cancer cells from the subject, treating the sample with on or more candidate treatments ex vivo, and determining whether the response of the cancer cells in the sample is consistent with clinical efficacy in vivo. Those treatments identified to have therapeutic potential can then be used in the screening method with the animal model of the invention.

Optionally, in the aforementioned screening method, the candidate treatment may be one previously determined to have efficacy in the treatment of at least some cancers in at least some patients or patient populations. Alternatively, the method may be aimed at drug discovery, in which the candidate treatment has not previously been identified to have efficacy in the treatment of cancer in vivo. Thus, if the objective is drug discovery, as opposed to the identification of an effective treatment for any single cancer, the method is a method for screening potential cancer treatments, comprising implanting one or more primary human cancer cells in or on the liver of a plurality of non-human animals; administering a plurality of candidate treatments to the animals before, during, or after said implanting; and evaluating at least one parameter of the one or more implanted primary human cancer cells and/or the animal that is associated with cancer treatment efficacy or lack of efficacy. The screening method can be carried out in parallel with multiple types of cancers and multiple candidate treatments, in high throughput fashion.

Another aspect of the invention includes a method for treating cancer in a subject, comprising selecting a candidate treatment from among a plurality of candidate treatments, and administering the selected treatment to the subject, wherein the selected candidate treatment has been determined to be effective in treating the cancer in a non-human animal model having one or more primary cancer cells from the cancer implanted in or on the liver of the animal.

In some embodiments, the methods of the invention further comprise harvesting the propagated cancer cells from the animal after the cells have been allowed to propagate in the animal. Optionally, cancer cells harvested from the animal may be placed in storage. In some embodiments, the method further comprises culturing (expanding) the harvested cancer cells and, optionally, storing the harvested cells. Optionally, harvested cancer cells may be cultured and/or stored and one or more of the cultured and/or stored cancer cells may be implanted in or on the liver of one or more other non-human animals. This process may be carried out repeatedly—in series, in parallel, or both. “Libraries” of animal models and cancer cells (e.g., tumor tissues) grown in animal models of the invention can be prepared and characterized based on biomarkers of the subject they were obtained from, based on biomarkers of the cancer cells themselves, and based on cancer treatments that the cancer cells are sensitive to or resistant to.

### III. Biomarker Discovery and Validation

Another aspect of the invention concerns a method for identifying a biomarker for cancer treatment, comprising providing a plurality of non-human animals having one or more primary human cancer cells from a plurality of humans implanted in or on the liver of the plurality of animals; administering a cancer treatment to the plurality of animals; identifying animals among the plurality of animals in which the cancer treatment is effective; and identifying a biomarker that is common to the identified animals and is associated with (correlates with) the treatment's effectiveness.

Another aspect of the invention concerns a method for identifying a biomarker for cancer treatment, comprising providing a plurality of non-human animals having one or more primary human cancer cells from a plurality of humans implanted in or on the liver of the plurality of animals; administering a cancer treatment to the plurality of animals; identifying animals among the plurality of animals in which the cancer treatment is ineffective; and identifying a biomarker that is common to the identified animals and is associated with (conelates with) the treatment's ineffectiveness.

### IV. Clinical Trial Enrichment: Patient Stratification and Clinical Trial Design

Another aspect of the invention concerns a method for selecting cancer patients for a clinical trial, comprising providing a plurality of non-human animals having one or more primary human cancer cells from a plurality of humans implanted in or on the liver of the plurality of animals; administering a cancer treatment to the plurality of animals; identifying animals among the plurality of animals in which the cancer treatment is effective; identifying a biomarker that is common to the identified animals and is associated with (correlates with) the treatment's effectiveness; and including (e.g., enrolling) a patient in the clinical trial if the patient has the biomarker or excluding the patient from the clinical if the patient lacks the biomarker.

Another aspect of the invention pertains to a method for selecting cancer patients for a clinical trial, comprising providing a plurality of non-human animals having one or more primary human cancer cells from a plurality of humans implanted in or on the liver of the plurality of animals; administering a cancer treatment to the plurality of animals; identifying animals among the plurality of animals in which the cancer treatment is ineffective; identifying a biomarker that is common to the identified animals and is associated with (conelates with) the treatment's ineffectiveness; and including (e.g., enrolling) a patient in the clinical trial if the patient lacks the biomarker or excluding the patient from the clinical trial if the patient has the biomarker.

### EXEMPLIFIED EMBODIMENTS

### Embodiment 1

An animal model comprising a non-human animal having one or more primary human cancer cells implanted in or on the liver of said animal.

### Embodiment 2

The animal model of embodiment 1, wherein the one or more human cancer cells are obtained directly from a human tumor (e.g., biopsy material).

### Embodiment 3

The animal model of embodiments 1 or 2, wherein the one or more human cancer cells are cells of a primary culture.

### Embodiment 5

The animal model of embodiment 1 or 2, wherein the primary human cancer cells are implanted in the liver.

### Embodiment 6

The animal model of embodiment 1, wherein the one or more human cancer cells comprises a plurality of primary human cancer cells, and wherein tumor tissue comprising the plurality of human cancer cells is implanted in or on the liver of the animal.

### Embodiment 7

The animal model of any preceding embodiment, wherein the one or more human cancer cells exhibit a state of growth (propagation) in or on the liver.

### Embodiment 8

The animal model of any one of embodiments 1-6, wherein the one or more human cancer cells originate from a primary tumor.

### Embodiment 9

The animal model of any one of embodiments 1-6, wherein the one or more human cancer cells originate from a metastatic tumor.

### Embodiment 10

The animal model of embodiment 9, wherein the one or more human cancer cells originated outside the human liver and metastasized to the human liver.

### Embodiment 11

The animal model of any preceding embodiment, wherein the one or more human cancer cells are heterotopic.

### Embodiment 12

The animal model of any one of embodiments 1-8, wherein the one or more human cancer cells are orthotopic.

### Embodiment 13

The animal model of any preceding embodiment, wherein the one or more human cancer cells bear a detectable label (e.g., a bioluminescent label such as luciferase).

### Embodiment 14

The animal model of any preceding embodiment, wherein the one or more human cancer cells carry a heterologous nucleic acid.

### Embodiment 15

The animal model of any preceding embodiment, wherein the animal is a rodent.

### Embodiment 16

The animal model of embodiment 15, wherein the rodent is a mouse.

### Embodiment 17

The animal model of any preceding embodiment, wherein the animal is immunodeficient.

### Embodiment 18

The animal model of any preceding embodiment, wherein the animal is genetically engineered.

### Embodiment 19

A method of producing the animal model of any one of embodiments 1-18, comprising implanting one or more primary human cancer cells in or on the liver of a non-human animal.

### Embodiment 20

A method of propagating human cancer cells, comprising implanting one or more primary human cancer cells in or on the liver of a non-human animal; and allowing the implanted cells to propagate.

### Embodiment 21

The method of embodiment 20, wherein implanting comprising making an incision in the liver of the animal, placing the one or more human cancer cells into the incision, and sealing the incision using a hemostatic patch or other hemostatic agent.

### Embodiment 22

The method of embodiment 20, wherein the one or more human cancer cells are obtained directly from a human tumor (e.g., biopsy material).

### Embodiment 23

The method of embodiment 20, wherein the one or more human cancer cells are cells of a primary culture.

### Embodiment 24

The method of any one of embodiments 20-23, further comprising harvesting the propagated cells from the animal.

### Embodiment 25

The method of embodiment 24, further comprising evaluating at least one parameter of the harvested cells (e.g., proteomic analysis, genomic analysis, analytical analysis).

### Embodiment 26

A method of evaluating human cancer cell growth, comprising providing the animal model of any one of embodiments 1-18, and evaluating the growth of the one or more primary human cancer cells in or on the liver of the animal.

### Embodiment 27

The method of embodiment 26, wherein the evaluating step is carried out in vivo with an imaging modality selected from among one or more of bioluminescent imaging (e.g., luciferase), ultrasound imaging, fluorescence molecular tomography (FMT), and magnetic resonance imaging (e.g., anatomical MRI, diffusion MRI, MRI spectroscopy, or dynamic contrast enhanced (DCE) MRI).

### Embodiment 28

The method of embodiment 26 or 27, wherein the evaluating comprises evaluating the growth of the one or more human cancer cells in response to a biologically active agent.

### Embodiment 29

The method of embodiment 26, wherein the biologically active agent comprises a combination of biologically active agents.

### Embodiment 30

The method of embodiment 26 or 27, wherein the biologically active agent comprises a chemotherapeutic or other anti-cancer agent.

### Embodiment 31

The method of embodiment 30, further comprising recording the sensitivity/resistance of the one or more human cancer cells to the anti-cancer agent in a computer readable medium.

### Embodiment 32

A method of studying human cancer, comprising providing the animal model of any one of embodiments 1-18, and evaluating at least one parameter of the one or more primary human cancer cells and/or the animal model.

### Embodiment 33

The method of embodiment 32, wherein the at least one parameter comprises the expression of a biomarker.

### Embodiment 34

The method of embodiment 33, wherein the biomarker comprises one or more tumor markers.

### Embodiment 35

The method of embodiment 32 or 33, further comprising recording the expression level of the biomarker in a computer readable medium.

### Embodiment 36

A method for screening potential treatments for a cancer in a human subject, comprising implanting one or more primary human cancer cells from the subject in or on the liver of a non-human animal; administering a candidate treatment to the animal before, during, or after said implanting; and evaluating at least one parameter of the one or more implanted primary human cancer cells and/or the animal that is associated with cancer treatment efficacy or lack of efficacy.

### Embodiment 37

The method of embodiment 36, wherein the candidate treatment comprises a chemotherapeutic or other anti-cancer agent.

### Embodiment 38

The method of embodiment 36, wherein the candidate treatment comprises a radiation treatment.

### Embodiment 39

The method of any one of embodiments 36-38, wherein the candidate treatment comprises a combination of treatments.

### Embodiment 40

The method of any one of embodiments 36-39, wherein the at least one parameter comprises cancer cell growth rate or tumor size.

### Embodiment 41

The method of any one of embodiments 39-40, wherein the evaluating step comprises imaging at least a portion of the animal to determine the response of the one or more human cancer cells to the candidate treatment.

### Embodiment 42

The method of any one of embodiments 36-41, wherein the implanting step comprises implanting one or more primary human cancer cells from the subject in or on the liver of a plurality of non-human animals, and wherein the administering step comprises administering a candidate treatment to each animal before, during, or after said implanting.

### Embodiment 43

The method of embodiment 42, wherein a different candidate treatment is administered to each animal.

### Embodiment 44

The method of embodiment 42, wherein a different dose of the same candidate treatment is administered to each animal.

### Embodiment 45

The method of any one of embodiments 36-44, further comprising selecting and administering the candidate treatment to the subject if the results of the evaluating step are consistent with cancer treatment efficacy.

### Embodiment 46

The method of any one of embodiments 36-45, wherein implanting comprising making an incision in the liver of the animal, placing the one or more human cancer cells into the incision, and sealing the incision using a hemostatic patch or other hemostatic agent.

### Embodiment 47

The method of any one of embodiments 36-46, wherein the one or more human cancer cells are metastatic cancer cells.

### Embodiment 48

The method of any one of embodiments 36-47, wherein the one or more human cancer cells originated outside the human liver and metastasized to the human liver.

### Embodiment 49

A method for treating cancer in a human subject, comprising selecting a candidate treatment from among a plurality of candidate treatments, and administering the selected treatment to the subject, wherein the selected candidate treatment has been determined to be effective in treating the cancer in a non-human animal model having one or more primary cancer cells from the cancer implanted in or on the liver of the animal.

### Embodiment 50

A method for screening potential cancer treatments, comprising implanting one or more primary human cancer cells in or on the liver of a plurality of non-human animals; administering a plurality of candidate treatments to the animals before, during, or after the implanting; and evaluating at least one parameter of the one or more implanted primary human cancer cells and/or the animal that is associated with cancer treatment efficacy or lack of efficacy.

### Embodiment 51

A method for identifying a biomarker for cancer treatment, comprising providing a plurality of non-human animals having one or more primary human cancer cells from a plurality of humans implanted in or on the liver of the plurality of animals; administering a cancer treatment to the plurality of animals; identifying animals among the plurality of animals in which the cancer treatment is effective; and identifying a biomarker that is common to the identified animals and is associated with (correlates with) the treatment's effectiveness.

### Embodiment 52

A method for identifying a biomarker for cancer treatment, comprising providing a plurality of non-human animals having one or more primary human cancer cells from a plurality of humans implanted in or on the liver of the plurality of animals; administering a cancer treatment to the plurality of animals; identifying animals among the plurality of animals in which the cancer treatment is ineffective; and identifying a biomarker that is common to the identified animals and is associated with (correlates with) the treatment's ineffectiveness.

### Embodiment 53

A method for selecting cancer patients for a clinical trial, comprising providing a plurality of non-human animals having one or more primary human cancer cells from a plurality of humans implanted in or on the liver of the plurality of animals; administering a cancer treatment to the plurality of animals; identifying animals among the plurality of animals in which the cancer treatment is effective; identifying a biomarker that is common to the identified animals and is associated with (correlates with) the treatment's effectiveness; and including a patient in the clinical trial if the patient has the biomarker or excluding the patient from the clinical trial if the patient lacks the biomarker.

### Embodiment 54

A method for selecting cancer patients for a clinical trial, comprising providing a plurality of non-human animals having one or more primary human cancer cells from a plurality of humans implanted in or on the liver of the plurality of animals; administering a cancer treatment to the plurality of animals; identifying animals among the plurality of animals in which the cancer treatment is ineffective; identifying a biomarker that is common to the identified animals and is associated with (correlates with) the treatment's ineffectiveness; and including a patient in the clinical trial if the patient lacks the biomarker or excluding the patient from the clinical trial if the patient has the biomarker.

### Embodiment 55

An animal model comprising a non-human animal having one or more primary cancer cells implanted in or on the liver of said animal, wherein the primary cancer cells are from a species different from that of the animal.

### Embodiment 56

The animal model of embodiment 55, wherein the primary cancer cells are from a non-human species.

As used herein, the term “primary cancer cells” means cancer cells other than cells of a cell line. The primary cancer cells may be obtained, for example, directly from a tumor (solid tumor or non-solid tumor), such as biopsy material, cancer cells obtained from a primary culture, or cancer cells passaged a limited number of times (e.g., passaged one, two, or three times). In some embodiments, the primary cancer cells are a cell strain (cells adapted to culture, but with finite division potential). A cell line is a permanently established cell culture that will proliferate indefinitely, given appropriate fresh medium and space. Cell lines differ from cell strains in that they have escaped the Hayflick limit (the number of times a normal cell population will divide before it stops (e.g., forty to sixty times) and become immortalized (Shay et al., Nat. Rev. Molec. Cell Biol., 2000, 1(1):72-76).

As used herein, the terms “immune deficiency” and “immune deficient” refer to a condition under which: a portion or some portions of cell components constituting an immune system are defective or dysfunction, so that a normal immune mechanism is damaged. In other words, the terms “immune deficiency” and “immune deficient” refer to a condition under which congenital immunity and/or acquired immunity are suppressed so that the one or more primary cancer cells are engrafted into an animal. An immune deficient animal is an immuno-compromised animal.

As used herein, the term “biomarker” refers to the presence or absence of a characteristic or trait (e.g., gene expression signature, gene expression score, life style factor, patient history) that is either consistent with a favorable clinical response (e.g., increased survival, decreased tumor size) or inconsistent with a favorable clinical response of a subject (human or non-human animal) to a treatment under study, i.e., is a determinant of response to a treatment. Biomarkers may be qualitative and/or quantitative. In some embodiments, the biomarker is a molecular biomarker (i.e., a molecular determinant of treatment response) such as a variation in a nucleic acid sequence or nucleic acid level (e.g., a single nucleotide polymorphism (SNP), or microRNA), a variation in a polypeptide sequence or level, or combinations of specific biochemical changes. For example, a biomarker may be a variation (increase or decrease) in the level of a signaling molecule or member of a signal transduction pathway. Preferably, the presence or absence of the characteristic is determined to correlate with the favorable clinical response at a statistically significant level or determined to correlate with an unfavorable clinical response at a statistically significant level. Methods for identifying biomarkers and measuring biomarkers in samples in vitro and in tissues in vivo are known in the art and may be used in carrying out the methods of the invention. In some embodiments, biomarkers of the primary cancer cells and/or the recipient animal are detected/measured before and after implantation of the primary cancer cells. In the various embodiments in the methods of the invention, biomarkers may be determined, detected, and measured within cancer cells themselves, within the subject from which the cancer cells were obtained, and within the animal model in which cancer cells are implanted.

The terms “cancer” and “malignancy” are used herein interchangeably to refer to or describe the physiological condition in mammals that is typically characterized by unregulated cell growth. The one or more primary cancer cells implanted in the animals in accordance with the invention can be that of any cancer type. The cancer may be drug-resistant (e.g., chemoresistant) or drug-sensitive. Examples of cancer include but are not limited to, carcinoma, lymphoma, blastoma, sarcoma, and leukemia. More particular examples of cancers include breast cancer, prostate cancer, colon cancer, squamous cell cancer, small-cell lung cancer, non-small cell lung cancer, gastrointestinal cancer, pancreatic cancer, cervical cancer, ovarian cancer, peritoneal cancer, liver cancer, e.g., hepatic carcinoma, bladder cancer, colorectal cancer, endometrial carcinoma, kidney cancer, and thyroid cancer.

Other non-limiting examples of cancers are basal cell carcinoma, biliary tract cancer; bone cancer; brain and CNS cancer; choriocarcinoma; connective tissue cancer; esophageal cancer; eye cancer; cancer of the head and neck; gastric cancer; intra-epithelial neoplasm; larynx cancer; lymphoma including Hodgkin's and Non-Hodgkin's lymphoma; melanoma; myeloma; neuroblastoma; oral cavity cancer (e.g., lip, tongue, mouth, and pharynx); retinoblastoma; rhabdomyosarcoma; rectal cancer; cancer of the respiratory system; sarcoma; skin cancer; stomach cancer; testicular cancer; uterine cancer; cancer of the urinary system, as well as other carcinomas and sarcomas. A non-exhaustive list of cancer types are also listed in Table 1. Other examples of cancer cell types that may be used with the animal models and methods of the invention include those listed in U.S. Patent Publication 2009/0325202 (for example, in paragraph [0080]), which is incorporated herein by reference in its entirety.

TABLE 1
Examples of Cancer Types

| | |
|---|---|
| Acute Lymphoblastic Leukemia, Adult | Hairy Cell Leukemia |
| Acute Lymphoblastic Leukemia, | Head and Neck Cancer |
| Childhood | Hepatocellular (Liver) Cancer, Adult (Primary) |
| Acute Myeloid Leukemia, Adult | Hepatocellular (Liver) Cancer, Childhood |
| Acute Myeloid Leukemia, Childhood | (Primary) |
| Adrenocortical Carcinoma | Hodgkin's Lymphoma, Adult |
| Adrenocortical Carcinoma, Childhood | Hodgkin's Lymphoma, Childhood |
| AIDS-Related Cancers | Hodgkin's Lymphoma During Pregnancy |
| AIDS-Related Lymphoma | Hypopharyngeal Cancer |
| Anal Cancer | Hypothalamic and Visual Pathway Glioma, |
| Astrocytoma, Childhood Cerebellar | Childhood |
| Astrocytoma, Childhood Cerebral | Intraocular Melanoma |
| Basal Cell Carcinoma | Islet Cell Carcinoma (Endocrine Pancreas) |
| Bile Duct Cancer, Extrahepatic | Kaposi's Sarcoma |
| Bladder Cancer | Kidney (Renal Cell) Cancer |
| Bladder Cancer, Childhood | Kidney Cancer, Childhood |
| Bone Cancer, Osteosarcoma/Malignant | Laryngeal Cancer |
| Fibrous Histiocytoma | Laryngeal Cancer, Childhood |
| Brain Stem Glioma, Childhood | Leukemia, Acute Lymphoblastic, Adult |
| Brain Tumor, Adult | Leukemia, Acute Lymphoblastic, Childhood |
| Brain Tumor, Brain Stem Glioma, | Leukemia, Acute Myeloid, Adult |
| Childhood | Leukemia, Acute Myeloid, Childhood |
| Brain Tumor, Cerebellar Astrocytoma, | Leukemia, Chronic Lymphocytic |
| Childhood | Leukemia, Chronic Myelogenous |
| Brain Tumor, Cerebral | Leukemia, Hairy Cell |
| Astrocytoma/Malignant Glioma, | Lip and Oral Cavity Cancer |
| Childhood | Liver Cancer, Adult (Primary) |
| Brain Tumor, Ependymoma, Childhood | Liver Cancer, Childhood (Primary) |
| Brain Tumor, Medulloblastoma, | Lung Cancer, Non-Small Cell |
| Childhood | Lung Cancer, Small Cell |
| Brain Tumor, Supratentorial Primitive | Lymphoma, AIDS-Related |
| Neuroectodermal Tumors, Childhood | Lymphoma, Burkitt's |
| Brain Tumor, Visual Pathway and | Lymphoma, Cutaneous T-Cell, see Mycosis |
| Hypothalamic Glioma, Childhood | Fungoides and Sézary Syndrome |
| Brain Tumor, Childhood | Lymphoma, Hodgkin's, Adult |
| Breast Cancer | Lymphoma, Hodgkin's, Childhood |
| Breast Cancer, Childhood | Lymphoma, Hodgkin's During Pregnancy |
| Breast Cancer, Male | Lymphoma, Non-Hodgkin's, Adult |
| Bronchial Adenomas/Carcinoids, | Lymphoma, Non-Hodgkin's, Childhood |
| Childhood | Lymphoma, Non-Hodgkin's During Pregnancy |
| Burkitt's Lymphoma | Lymphoma, Primary Central Nervous System |
| Carcinoid Tumor, Childhood | Macroglobulinemia, Waldenström's |
| Carcinoid Tumor, Gastrointestinal | Malignant Fibrous Histiocytoma of |
| Carcinoma of Unknown Primary | Bone/Osteosarcoma |
| Central Nervous System Lymphoma, | Medulloblastoma, Childhood |
| Primary | Melanoma |
| Cerebellar Astrocytoma, Childhood | Melanoma, Intraocular (Eye) |
| Cerebral Astrocytoma/Malignant Glioma, | Merkel Cell Carcinoma |
| Childhood | Mesothelioma, Adult Malignant |
| Cervical Cancer | Mesothelioma, Childhood |
| Childhood Cancers | Metastatic Squamous Neck Cancer with Occult |
| Chronic Lymphocytic Leukemia | Primary |
| Chronic Myelogenous Leukemia | Multiple Endocrine Neoplasia Syndrome, |
| Chronic Myeloproliferative Disorders | Childhood |
| Colon Cancer | Multiple Myeloma/Plasma Cell Neoplasm |
| Colorectal Cancer, Childhood | Mycosis Fungoides |
| Cutaneous T-Cell Lymphoma, see | Myelodysplastic Syndromes |
| Mycosis Fungoides and Sézary | Myelodysplastic/Myeloproliferative Diseases |
| Syndrome | Myelogenous Leukemia, Chronic |
| Endometrial Cancer | Myeloid Leukemia, Adult Acute |
| Ependymoma, Childhood | Myeloid Leukemia, Childhood Acute |
| Esophageal Cancer | Myeloma, Multiple |
| Esophageal Cancer, Childhood | Myeloproliferative Disorders, Chronic |
| Ewing's Family of Tumors | Nasal Cavity and Paranasal Sinus Cancer |
| Extracranial Germ Cell Tumor, | Nasopharyngeal Cancer |
| Childhood | Nasopharyngeal Cancer, Childhood |
| Extragonadal Germ Cell Tumor | Neuroblastoma |
| Extrahepatic Bile Duct Cancer | Non-Hodgkin's Lymphoma, Adult |
| Eye Cancer, Intraocular Melanoma | Non-Hodgkin's Lymphoma, Childhood |
| Eye Cancer, Retinoblastoma | Non-Hodgkin's Lymphoma During Pregnancy |
| Gallbladder Cancer | Non-Small Cell Lung Cancer |
| Gastric (Stomach) Cancer | Oral Cancer, Childhood |
| Gastric (Stomach) Cancer, Childhood | Oral Cavity Cancer, Lip and |
| Gastrointestinal Carcinoid Tumor | Oropharyngeal Cancer |
| Germ Cell Tumor, Extracranial, | Osteosarcoma/Malignant Fibrous Histiocytoma |
| Childhood | of Bone |
| Germ Cell Tumor, Extragonadal | Ovarian Cancer, Childhood |
| Germ Cell Tumor, Ovarian | Ovarian Epithelial Cancer |
| Gestational Trophoblastic Tumor | Ovarian Germ Cell Tumor |
| Glioma, Adult | Ovarian Low Malignant Potential Tumor |
| Glioma, Childhood Brain Stem | Pancreatic Cancer |
| Glioma, Childhood Cerebral | Pancreatic Cancer, Childhood |
| Astrocytoma | Pancreatic Cancer, Islet Cell |
| Glioma, Childhood Visual Pathway and | Paranasal Sinus and Nasal Cavity Cancer |
| Hypothalamic | Parathyroid Cancer |
| Skin Cancer (Melanoma) | Penile Cancer |
| Skin Carcinoma, Merkel Cell | Pheochromocytoma |
| Small Cell Lung Cancer | Pineoblastoma and Supratentorial Primitive |
| Small Intestine Cancer | Neuroectodermal Tumors, Childhood |
| Soft Tissue Sarcoma, Adult | Pituitary Tumor |
| Soft Tissue Sarcoma, Childhood | Plasma Cell Neoplasm/Multiple Myeloma |
| Squamous Cell Carcinoma, see Skin | Pleuropulmonary Blastoma |
| Cancer (non-Melanoma) | Pregnancy and Breast Cancer |
| Squamous Neck Cancer with Occult | Pregnancy and Hodgkin's Lymphoma |
| Primary, Metastatic | Pregnancy and Non-Hodgkin's Lymphoma |
| Stomach (Gastric) Cancer | Primary Central Nervous System Lymphoma |
| Stomach (Gastric) Cancer, Childhood | Prostate Cancer |
| Supratentorial Primitive | Rectal Cancer |
| Neuroectodermal Tumors, Childhood | Renal Cell (Kidney) Cancer |
| T-Cell Lymphoma, Cutaneous, see | Renal Cell (Kidney) Cancer, Childhood |
| Mycosis Fungoides and Sézary | Renal Pelvis and Ureter, Transitional Cell |
| Syndrome | Cancer |
| Testicular Cancer | Retinoblastoma |
| Thymoma, Childhood | Rhabdomyosarcoma, Childhood |
| Thymoma and Thymic Carcinoma | Salivary Gland Cancer |
| Thyroid Cancer | Salivary Gland Cancer, Childhood |
| Thyroid Cancer, Childhood | Sarcoma, Ewing's Family of Tumors |
| Transitional Cell Cancer of the Renal | Sarcoma, Kaposi's |
| Pelvis and Ureter | Sarcoma, Soft Tissue, Adult |
| Trophoblastic Tumor, Gestational | Sarcoma, Soft Tissue, Childhood |
| Unknown Primary Site, Carcinoma of, | Sarcoma, Uterine |
| Adult | Sezary Syndrome |
| Unknown Primary Site, Cancer of, | Skin Cancer (non-Melanoma) |
| Childhood | Skin Cancer, Childhood |
| Unusual Cancers of Childhood | |
| Ureter and Renal Pelvis, Transitional Cell | |
| Cancer | |
| Urethral Cancer | |
| Uterine Cancer, Endometrial | |
| Uterine Sarcoma | |
| Vaginal Cancer | |
| Visual Pathway and Hypothalamic | |
| Glioma, Childhood | |
| Vulvar Cancer | |
| Waldenström's Macroglobulinemia | |
| Wilms' Tumor | |

As used herein, the terms “administering” or “administer” are defined as the introduction of a substance (such as biologically active agents) into cells in vitro or into the body of a human or non-human animal subject in vivo by any route (for example, oral, nasal, ocular, rectal, vaginal and parenteral routes). Substances may be administered individually or in combination with other agents via any route of administration, including but not limited to subcutaneous (SQ), intramuscular (IM), intravenous (IV), intraperitoneal (IP), intradermal (ID), via the nasal, ocular or oral mucosa (IN), or orally. For example, substances can be administered by direct injection into or on a tumor, or systemically (e.g., into the circulatory system), to kill circulating tumor cells (CTC). “Implantation” refers to the administration of cells (e.g., one or more primary cancer cells) in vivo. Any implantation technique effective in delivering the cells to the target anatomical site (e.g., liver) can be utilized. For example, the cells can be implanted in or on the liver in an open surgical manner or through a catheter (e.g., intrahepatic injection).

As used herein, the terms “treat” or “treatment” refer to both therapeutic treatment and prophylactic or preventative measures, wherein the object is to prevent or slow down (lessen) an undesired physiological change or disorder, such as the development or spread of cancer, reduce the growth of cancer cells, reduce tumor size, inhibit tumor growth, etc. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. For example, treatment may include reduction of undesirable cell proliferation, and/or induction of apoptosis and cytotoxicity. “Treatment” can also mean prolonging survival as compared to expected survival if not receiving treatment. Those in need of treatment include those already with the condition or disorder as well as those prone to have the condition or disorder or those in which the condition or disorder is to be prevented or onset delayed. Optionally, the subject may be identified (e.g., diagnosed) as one suffering from the disease or condition (e.g., cancer) prior to administration of the treatment. Examples of treatment include but are not limited to, chemotherapy, radiation therapy, immunotherapy, or a combination of two or more of the foregoing. A “candidate treatment” may be a treatment that has been previously identified to have efficacy in treating at least some cancer types in vitro or in vivo, or the candidate treatment may have no known efficacy in treating cancer.

As used herein, the term “(therapeutically) effective amount” refers to an amount of a treatment (e.g., anticancer agent) to treat a disease or disorder in a human or non-human animal subject. In the case of cancer or other proliferation disorder, the therapeutically effective amount of the treatment may reduce (i.e., slow to some extent and preferably stop) unwanted cellular proliferation; reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve, to some extent, one or more of the symptoms associated with the cancer. To the extent the administered treatment prevents growth of and/or kills existing cancer cells, it may be cytostatic and/or cytotoxic. For cancer therapy, efficacy can, for example, be measured by assessing the time to disease progression (TTP) and/or determining the response rate (RR).

As used herein, the term “growth inhibitory amount” of the treatment refers to an amount which inhibits growth or proliferation of a target cell, such as a tumor cell, either in vitro or in vivo, irrespective of the mechanism by which cell growth is inhibited (e.g., by cytostatic properties, cytotoxic properties, etc.). In a preferred embodiment, the growth inhibitory amount inhibits (i.e., slows to some extent and preferably stops) proliferation or growth of the target cell in vivo (e.g., in an animal model of the invention) or in vitro (e.g., in cell culture) by greater than about 20%, preferably greater than about 50%, most preferably greater than about 75% (e.g., from about 75% to about 100%). The animal models of the invention may be used to determine the growth inhibitory amount of a treatment (e.g., growth inhibitory amount of a chemotherapeutic or other anti-cancer agent) for a particular subject's cancer. For example, following implantation of one or more primary cancer cells from a subject in or on the liver of the animal model, the treatment can be administered to the animal, and the growth inhibitory amount of the treatment can be determined in vivo.

The terms “cell” and “cells” are used interchangeably herein and are intended to include either a single cell or a plurality of cells, in vitro or in vivo, unless otherwise specified.

As used herein, the term “anti-cancer agent” refers to a substance or treatment (e.g., radiation therapy) that inhibits the function of cancer cells, inhibits their formation, and/or causes their destruction in vitro or in vivo. Examples include, but are not limited to, cytotoxic agents (e.g., 5-fluorouracil, TAXOL), chemotherapeutic agents, and anti-signaling agents (e.g., the PI3K inhibitor LY). In some embodiments of some methods of the invention, an anti-cancer agent is administered to an animal model of the invention before, during, after implantation of one or more primary cancer cells in or on the liver of the animal. Anti-cancer agents include but are not limited to the chemotherapeutic agents listed Table 2.

As used herein, the term “cytotoxic agent” refers to a substance that inhibits or prevents the function of cells and/or causes destruction of cells in vitro and/or in vivo. The term is intended to include radioactive isotopes (e.g., Ar²¹¹, I¹³¹, I¹²⁵, Y⁹⁰, Re¹⁸⁶, Re¹⁸⁸, Sm¹⁵³, Bi²¹², P³², and radioactive isotopes of Lu), chemotherapeutic agents, toxins such as small molecule toxins or enzymatically active toxins of bacterial, fungal, plant or animal origin, and antibodies, including fragments and/or variants thereof.

As used herein, the term “chemotherapeutic agent” is a chemical compound useful in the treatment of cancer, such as, for example, taxanes, e.g., paclitaxel (TAXOL, BRISTOL-MYERS SQUIBB Oncology, Princeton, N.J.) and doxetaxel (TAXOTERE, Rhone-Poulenc Rorer, Antony, France), chlorambucil, vincristine, vinblastine, anti-estrogens including for example tamoxifen, raloxifene, aromatase inhibiting 4(5)-imidazoles, 4-hydroxytamoxifen, trioxifene, keoxifene, LY117018, onapristone, and toremifene (FARESTON, GTx, Memphis, Tenn.), and anti-androgens such as flutamide, nilutamide, bicalutamide, leuprolide, and goserelin, etc. Examples of chemotherapeutic agents that may be used in conjunction with the compounds of the invention are listed in Table 2. In some embodiments, the chemotherapeutic agent is one or more anthracyclines. Anthracyclines are a family of chemotherapy drugs that are also antibiotics. The anthracyclines act to prevent cell division by disrupting the structure of the DNA and terminate its function by: (1) intercalating into the base pairs in the DNA minor grooves; and (2) causing free radical damage of the ribose in the DNA. The anthracyclines are frequently used in leukemia therapy. Examples of anthracyclines include daunorubicin (CERUBIDINE), doxorubicin (ADRIAMYCIN, RUBEX), epirubicin (ELLENCE, PHARMORUBICIN), and idarubicin (IDAMYCIN). Other examples of agents that may be screened or assessed in accordance with the methods of the invention include the therapeutic agents listed in U.S. Patent Publication 2009/0325202 (for example, paragraphs [0113]-[0116]), which is incorporated herein by reference in its entirety.

TABLE 2
Examples of Chemotherapeutic Agents

| | |
|---|---|
| 13-cis-Retinoic Acid | Mylocel |
| 2-Amino-6- | Letrozole |
| Mercaptopurine | Neosar |
| 2-CdA | Neulasta |
| 2-Chlorodeoxyadenosine | Neumega |
| 5-fluorouracil | Neupogen |
| 5-FU | Nilandron |
| 6-TG | Nilutamide |
| 6-Thioguanine | Nitrogen Mustard |
| 6-Mercaptopurine | Novaldex |
| 6-MP | Novantrone |
| Accutane | Octreotide |
| Actinomycin-D | Octreotide acetate |
| Adriamycin | Oncospar |
| Adrucil | Oncovin |
| Agrylin | Ontak |
| Ala-Cort | Onxal |
| Aldesleukin | Oprevelkin |
| Alemtuzumab | Orapred |
| Alitretinoin | Orasone |
| Alkaban-AQ | Oxaliplatin |
| Alkeran | Paclitaxel |
| All-transretinoic acid | Pamidronate |
| Alpha interferon | Panretin |
| Altretamine | Paraplatin |
| Amethopterin | Pediapred |
| Amifostine | PEG Interferon |
| Aminoglutethimide | Pegaspargase |
| Anagrelide | Pegfilgrastim |
| Anandron | PEG-INTRON |
| Anastrozole | PEG-L-asparaginase |
| Arabinosylcytosine | Phenylalanine Mustard |
| Ara-C | Platinol |
| Aranesp | Platinol-AQ |
| Aredia | Prednisolone |
| Arimidex | Prednisone |
| Aromasin | Prelone |
| Arsenic trioxide | Procarbazine |
| Asparaginase | PROCRIT |
| ATRA | Proleukin |
| Avastin | Prolifeprospan 20 with Carmustine implant |
| BCG | Purinethol |
| BCNU | Raloxifene |
| Bevacizumab | Rheumatrex |
| Bexarotene | Rituxan |
| Bicalutamide | Rituximab |
| BiCNU | Roveron-A (interferon alfa-2a) |
| Blenoxane | Rubex |
| Bleomycin | Rubidomycin hydrochloride |
| Bortezomib | Sandostatin |
| Busulfan | Sandostatin LAR |
| Busulfex | Sargramostim |
| C225 | Solu-Cortef |
| Calcium Leucovorin | Solu-Medrol |
| Campath | STI-571 |
| Camptosar | Streptozocin |
| Camptothecin-11 | Tamoxifen |
| Capecitabine | Targretin |
| Carac | Taxol |
| Carboplatin | Taxotere |
| Carmustine | Temodar |
| Carmustine wafer | Temozolomide |
| Casodex | Teniposide |
| CCNU | TESPA |
| CDDP | Thalidomide |
| CeeNU | Thalomid |
| Cerubidine | TheraCys |
| cetuximab | Thioguanine |
| Chlorambucil | Thioguanine Tabloid |
| Cisplatin | Thiophosphoamide |
| Citrovorum Factor | Thioplex |
| Cladribine | Thiotepa |
| Cortisone | TICE |
| Cosmegen | Toposar |
| CPT-11 | Topotecan |
| Cyclophosphamide | Toremifene |
| Cytadren | Trastuzumab |
| Cytarabine | Tretinoin |
| Cytarabine liposomal | Trexall |
| Cytosar-U | Trisenox |
| Cytoxan | TSPA |
| Dacarbazine | VCR |
| Dactinomycin | Velban |
| Darbepoetin alfa | Velcade |
| Daunomycin | VePesid |
| Daunorubicin | Vesanoid |
| Daunorubicin | Viadur |
| hydrochloride | Vinblastine |
| Daunorubicin liposomal | Vinblastine Sulfate |
| DaunoXome | Vincasar Pfs |
| Decadron | Vincristine |
| Delta-Cortef | Vinorelbine |
| Deltasone | Vinorelbine tartrate |
| Denileukin diftitox | VLB |
| DepoCyt | VP-16 |
| Dexamethasone | Vumon |
| Dexamethasone acetate | Xeloda |
| dexamethasone sodium | Zanosar |
| phosphate | Zevalin |
| Dexasone | Zinecard |
| Dexrazoxane | Zoladex |
| DHAD | Zoledronic acid |
| DIC | Zometa |
| Diodex | Gliadel wafer |
| Docetaxel | Glivec |
| Doxil | GM-CSF |
| Doxorubicin | Goserelin |
| Doxorubicin liposomal | granulocyte-colony stimulating factor |
| Droxia | Granulocyte macrophage colony stimulating |
| DTIC | factor |
| DTIC-Dome | Halotestin |
| Duralone | Herceptin |
| Efudex | Hexadrol |
| Eligard | Hexalen |
| Ellence | Hexamethylmelamine |
| Eloxatin | HMM |
| Elspar | Hycamtin |
| Emcyt | Hydrea |
| Epirubicin | Hydrocort Acetate |
| Epoetin alfa | Hydrocortisone |
| Erbitux | Hydrocortisone sodium phosphate |
| Erwinia L-asparaginase | Hydrocortisone sodium succinate |
| Estramustine | Hydrocortone phosphate |
| Ethyol | Hydroxyurea |
| Etopophos | Ibritumomab |
| Etoposide | Ibritumomab Tiuxetan |
| Etoposide phosphate | Idamycin |
| Eulexin | Idarubicin |
| Evista | Ifex |
| Exemestane | IFN-alpha |
| Fareston | Ifosfamide |
| Faslodex | IL-2 |
| Femara | IL-11 |
| Filgrastim | Imatinib mesylate |
| Floxuridine | Imidazole Carboxamide |
| Fludara | Interferon alfa |
| Fludarabine | Interferon Alfa-2b (PEG conjugate) |
| Fluoroplex | Interleukin-2 |
| Fluorouracil | Interleukin-11 |
| Fluorouracil (cream) | Intron A (interferon alfa-2b) |
| Fluoxymesterone | Leucovorin |
| Flutamide | Leukeran |
| Folinic Acid | Leukine |
| FUDR | Leuprolide |
| Fulvestrant | Leurocristine |
| G-CSF | Leustatin |
| Gefitinib | Liposomal Ara-C |
| Gemcitabine | Liquid Pred |
| Gemtuzumab ozogamicin | Lomustine |
| Gemzar | L-PAM |
| Gleevec | L-Sarcolysin |
| Lupron | Meticorten |
| Lupron Depot | Mitomycin |
| Matulane | Mitomycin-C |
| Maxidex | Mitoxantrone |
| Mechlorethamine | M-Prednisol |
| Mechlorethamine | MTC |
| Hydrochlorine | MTX |
| Medralone | Mustargen |
| Medrol | Mustine |
| Megace | Mutamycin |
| Megestrol | Myleran |
| Megestrol Acetate | Iressa |
| Melphalan | Irinotecan |
| Mercaptopurine | Isotretinoin |
| Mesna | Kidrolase |
| Mesnex | Lanacort |
| Methotrexate | L-asparaginase |
| Methotrexate Sodium | LCR |
| Methylprednisolone | |

As used herein, the term “tumor” refers to all neoplastic cell growth and proliferation, whether malignant or benign, and all pre-cancerous and cancerous cells and tissues. For example, a particular cancer may be characterized by a solid tumor mass. A primary tumor mass refers to a growth of cancer cells in a tissue resulting from the transformation of a normal cell of that tissue. In most cases, the primary tumor mass is identified by the presence of a cyst, which can be found through visual or palpation methods, or by irregularity in shape, texture, or weight of the tissue. However, some primary tumors are not palpable and can be detected only through medical imaging techniques such as X-rays (e.g., mammography), or by needle aspirations. The use of these latter techniques is more common in early detection. Molecular and phenotypic analysis of cancer cells within a tissue will usually confirm if the cancer is endogenous to the tissue or if the lesion is due to metastasis from another site. Some cancer treatments function by inducing apoptosis in tumor cells and reducing tumor cell growth. Depending upon the treatment, some treatments can be administered locally at the site of a tumor (e.g., by direct injection) or remotely (e.g., systemically). As used herein, the term “tumor” includes all types of tumors, including solid tumors as well as non-solid tumor such as leukemia or other blood cancer. The term “tumor” includes not only primary tumors but also tumors formed by metastasization, such as organ metastases and bone marrow metastases, and cells from relapsing breast cancer tumors.

As used herein, the term “signaling” and “signaling transduction” represents the biochemical process involving transmission of extracellular stimuli, via cell surface receptors through a specific and sequential series of molecules, to genes in the nucleus resulting in specific cellular responses to the stimuli.

As used herein, the term “pharmaceutically acceptable salt or prodrug” is intended to describe any pharmaceutically acceptable form (such as an ester, phosphate ester, salt of an ester or a related group) of a biologically active molecule, which, upon administration to a subject, provides the mature or base compound. Pharmaceutically acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art. Pharmaceutically acceptable prodrugs refer to a compound that is metabolized, for example hydrolyzed or oxidized, in the host to Rhin the compound of the present invention. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound.

The primary cancer cells used in the invention may be labeled with a detectable label prior to implantation in or on the liver of the animal model. As used herein, the terms “label” and “tag” refer to substances that may confer a detectable signal, and include, but are not limited to, enzymes such as alkaline phosphatase, glucose-6-phosphate dehydrogenase, and horseradish peroxidase, ribozyme, a substrate for a replicase such as QB replicase, promoters, dyes, fluorescers, such as fluorescein, isothiocynate, rhodamine compounds, phycoerythrin, phycocyanin, allophycocyanin, o-phthaldehyde, and fluorescamine, chemiluminescers such as isoluminol, sensitizers, coenzymes, enzyme substrates, radiolabels, particles such as latex or carbon particles, liposomes, cells, etc., which may be further labeled with a dye, catalyst or other detectable group. In some embodiments, the label is a bioluminescent label such as luciferase.

The terms “comprising”, “consisting of” and “consisting essentially of” are defined according to their standard meaning. The terms may be substituted for one another throughout the instant application in order to attach the specific meaning associated with each term.

The terms “isolated” or “biologically pure” refer to material that is substantially or essentially free from components which normally accompany the material as it is found in its native state. Thus, isolated cells in accordance with the invention preferably do not contain materials normally associated with the cells in their in situ environment. For example, the one or more primary cancer cells implanted in or on the liver of the non-human animal in accordance with the invention may be in isolated form, or may include other cells and/or materials (as a crude specimen), at the time of implantation. Optionally, the one or more primary cancer cells may be purified or undergo selection techniques (e.g., using flow cytometry) in order to implant subsets of primary cancer cells, such as cancer stem cells.

As used in this specification, the singular forms “a”, “an”, and “the” include singular and plural reference unless the context clearly dictates otherwise. Thus, for example, a reference to “a compound” includes a single compound and more than one such compound. Reference to “cell” is inclusive of a single cell and more than one such cell. A reference to “a treatment” includes a single treatment and more than one such treatment, and so forth.

As used herein, the terms “subject”, “individual”, and “patient” are used interchangeably to refer to a human or non-human animal. In some embodiments, the subject is a mammal (human or non-human). In some embodiments, the subject is human. Subjects may be any age or gender.

The practice of the present invention can employ, unless otherwise indicated, conventional techniques of molecular biology, microbiology, recombinant DNA technology, electrophysiology, and pharmacology that are within the skill of the art. Such techniques are explained fully in the literature (see, e.g., Sambrook, Fritsch & Maniatis, Molecular Cloning: A Laboratory Manual, Second Edition (1989); DNA Cloning, Vols. I and II (D. N. Glover Ed. 1985); Perbal, B., A Practical Guide to Molecular Cloning (1984); the series, Methods In Enzymology (S. Colowick and N. Kaplan Eds., Academic Press, Inc.); Transcription and Translation (Hames et al. Eds. 1984); Gene Transfer Vectors For Mammalian Cells (J. H. Miller et al. Eds. (1987) Cold Spring Harbor Laboratory, Cold Spring Harbor, N.Y.); Scopes, Protein Purification Principles and Practice (2nd ed., Springer-Verlag); and PCR: A Practical Approach (McPherson et al. Eds. (1991) IRL Press)), each of which are incorporated herein by reference in their entirety.

Experimental controls are considered fundamental in experiments designed in accordance with the scientific method. It is routine in the art to use experimental controls in scientific experiments to prevent factors other than those being studied from affecting the outcome.

All patents, patent applications, provisional applications, and publications referred to or cited herein, supra or infra, are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### Example 1

### Implantation of Tumor Tissue Into Mouse Liver

Prepare before:
Sterile saline
18 g needles
10 ml syringes
1 ml syringes for IP saline and to rinse patch
100 mm plates
Hemostatic patches
50 ml conicals
Sterile scalpel blades (size 10 or smaller)
Sterile gloves

### Sterilize:

cotton swabs
surgical instruments (scissors, forceps, etc)
drapes
gauze
staples

### Prepare Donor Tissue:

1. Remove donor tumor and sterilely chop on ice into ˜3×3×3 mm chunks.
2. Spin 1500 rpm/5 min, shake gently to allow necrotic tissue to float to top aspirate supernatant and necrotic tissue.
3. Gently re-suspend healthy tissue chunks in fresh media and pour into sterile dish containing 1:1 ratio of Matrigel and media. Keep tumors on ice for 30 min to prepare for implantation.

### Liver Implantation:

4. Make midline incision of the skin and underlying fascia with scissor 1 cm (go wider rather then longer if necessary).
5. Carefully remove liver using sterile cotton swabs and rest on sterile cotton swab, drape or clean skin.
6. Make small incision in liver using 10 blade (0.5 cm long and 0.3 cm deep).
7. With either #5 forceps or 18 g needle onl Oml syringe pick up tumor chunks.
8. Push aggregates of 3-5 pieces deep in the liver with curved #5 forceps.
9. Place noninvasive hemostatic patch over incision, rinse with saline to activate for 1-2 minutes. Repeat if necessary.
10. Dab with sterile gauze to ensure bleeding stopped
11. Replace liver in the abdomen.
12. Staple incision site
13. add 0.1 ml ketoprofen (0.8 mg/ml)
14. add 0.2 ml saline IP to compensate for blood loss after waking

### Example 2

### Quantitative In Vivo Imaging of Patient-Derived Metastatic Tumor in the Mouse Liver

The quantitative fluorescence molecular tomography (FMT) systems provides non-invasive, whole body, fluorescence tomographic imaging for quantification of deep tissue targets in vivo. Using one or multiple targeted, activatable and vascular agents and labels (for multiplexed results), a broad range of biologic targets, biomarkers, pathways and processes in vivo can be analyzed in a quantitative manner.

The inventor utilized Integrisense-750 (PerkinElmer, Inc., Boston, Mass.) probe to detect expression of integrin αvβ3 protein in a patient tumor implanted in the mouse liver. For whole animal fluorescence imaging, mice were injected i.v in the tail vein with 10 μl per gram body weight of sterile prepared near-infrared fluorescent dye labeled molecular imaging probe. After 1 minute, animals were anesthetized with isoflurane and transferred to the thermo-regulated, dark chamber of the FMT 2500 small animal imaging system (PerkinElmer, Inc.). The system acquires and overlays photographic and luminescent images by using laser light to excite a fluorochrome and measuring the emitted fluorescence transmitted through the tissue. Proprietary software is used to analyze acquired images. Animals are kept warm under light isoflurane anesthesia throughout the procedure.

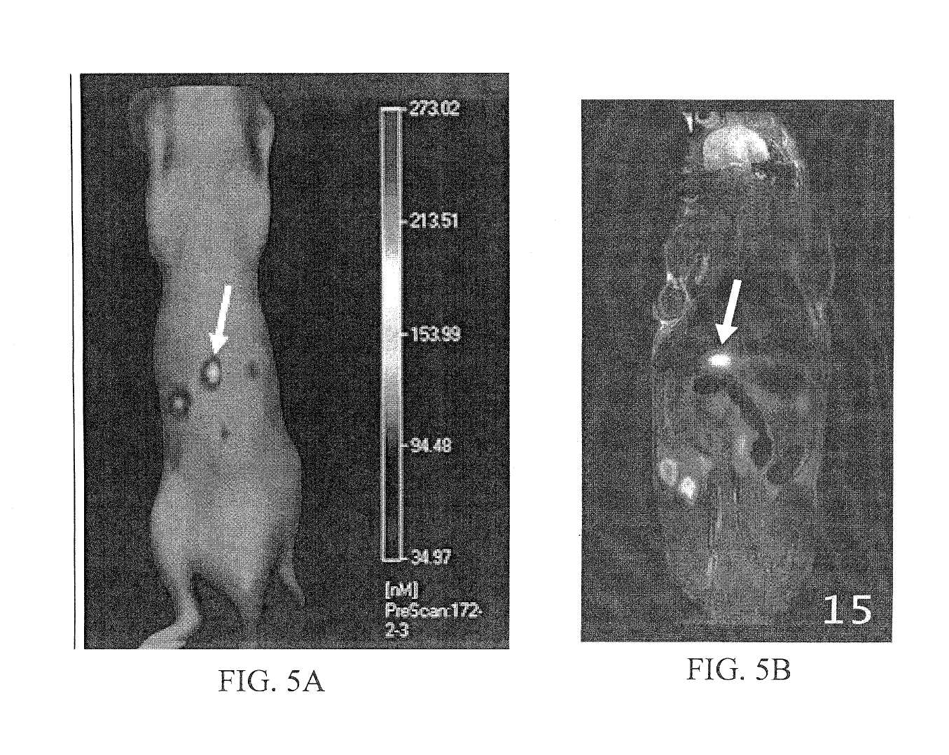

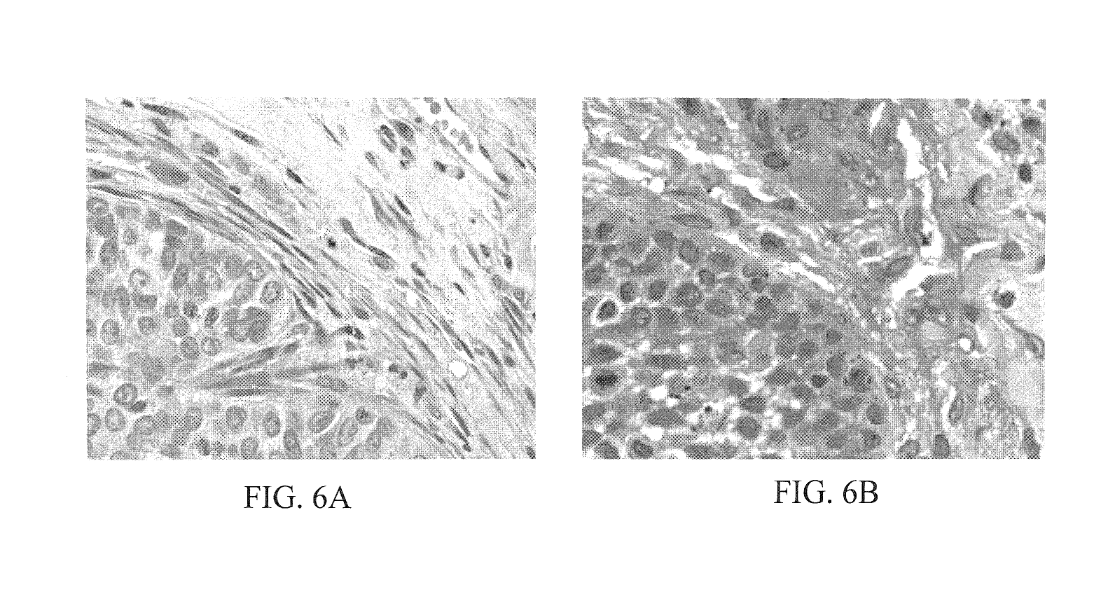

As illustrated in FIG. 5A, FMT imaging precisely located the tumor in the liver. Subsequently, the inventor performed MRI imaging with the same mouse, which corroborated tumor location (FIG. 5B). Histological sections of the tumor confirmed that the detected image was originated from the tumor (FIG. 6A), which was highly similar to the original patient's tumor (FIG. 6B).

This approach, utilizing FMT and/or other imaging modalities, can be used in orthotopic metastatic liver xenograft models to:
1. Monitor tumor growth and progression and therapeutic response in longitudinal mouse studies;
2. Integrate FMT in vivo data with in vitro readouts from microscopy and cell-based imaging;
3. Measure, monitor, and quantify biological targets, processes and pathways in vivo;
4. Obtain functional and biological data to enhance data and decision-making in research and drug development projects as well as in clinical studies to choose the most effective drug-drug combinations for individual patients; and
5. Perform in vivo imaging applications for bio-distribution, tumor targeting, drug/gene delivery, antibody and DNA-based tumor therapy as well as vascular and lymphatic imaging/targeting. For example, FMT may be combined with fluorescent-labeled drugs, peptides and other nanoparticles.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

1. An animal model comprising a non-human animal having one or more primary human cancer cells implanted in or on the liver of said animal.

2. The animal model of claim 1, wherein said one or more human cancer cells are obtained directly from a human tumor.

3. The animal model of claim 1, wherein said one or more human cancer cells are cells of a primary culture.

4. The animal model of claim 1, wherein the one or more human cancer cells originated outside the human liver and metastasized to the human liver.

5. The animal model of claim 1, wherein said one or more human cancer cells comprises a plurality of primary human cancer cells, and wherein tumor tissue comprising said plurality of human cancer cells is implanted in or on the liver of said animal.

6. The animal model of claim 1, wherein said one or more human cancer cells exhibit a state of growth (propagation) in or on the liver.

7. The animal model of claim 1, wherein said one or more human cancer cells originate from a primary tumor.

8. The animal model of claim 1, wherein said one or more human cancer cells originate from a metastatic tumor.

9. The animal model of claim 1, wherein said one or more human cancer cells are heterotopic.

10. The animal model of claim 1, wherein said one or more human cancer cells are orthotopic.

11. The animal model of claim 1, wherein said one or more human cancer cells bear a detectable label.

12. The animal model of claim 1, wherein said one or more human cancer cells carry a heterologous nucleic acid.

13. The animal model of claim 1, wherein said animal is a rodent.

14. The animal model of claim 13, wherein said rodent is a mouse.

15. The animal model of claim 1, wherein said animal is immunodeficient.

16. The animal model of claim 1, wherein said animal is genetically engineered.

17. A method selected from among:
(a) a method of producing an animal model, comprising implanting one or more primary cancer cells in or on the liver of a non-human animal; or
(b) a method of propa acing human cancer cells comprising implanting one or more primary human cancer cells in or on the liver of a non-human animal; and allowing the implanted cells to propagate; or
(c) a method of evaluating human cancer cell growth, comprising providing an animal model comprising a non-human animal having one or more primary human cancer cells implanted in or on the liver of said animal, and evaluating the growth of the one or more primary human cancer cells in or on the liver of said animal; or
(d) a method of studying human cancer, comprising providing an animal model comprising a non-human animal having one or more primary human cancer cells implanted in or on the liver of said animal, and evaluating at least one parameter of the one or more primary human cancer cells and/or the animal model; or
(e) a method for screening potential treatments for a cancer in a human subject, comprising implanting one or more primary human cancer cells from the subject in or on the liver of a non-human animal; administering a candidate treatment to the animal before, during, or after said implanting; and evaluating at least one parameter of the one or more implanted primary human cancer cells and/or the animal that is associated with cancer treatment efficacy or lack of efficacy; or
(f) a method for treating cancer in a human subject, comprising selecting a candidate treatment from among a plurality of candidate treatments, and administering the selected treatment to the subject, wherein the selected candidate treatment has been determined to be effective in treating the cancer in a non-human animal model having one or more primary cancer cells from the cancer implanted in or on the liver of the animal; or
(g) a method for screening potential cancer treatments, comprising implanting one or more primary human cancer cells in or on the liver of a plurality of non-human animals; administering a plurality of candidate treatments to the animals before, during, or after said implanting; and evaluating at least one parameter of the one or more implanted primary human cancer cells and/or the animal that is associated with cancer treatment efficacy or lack of efficacy; or
(h) a method for identifying a biomarker for cancer treatment, comprising providing a plurality of non-human animals having one or more primary human cancer cells from a plurality of humans implanted in or on the liver of the plurality of animals; administering a cancer treatment to the plurality of animals; identifying animals among the plurality of animals in which the cancer treatment is effective; and identifying a biomarker that is common to the identified animals and is associated with (correlates with) the treatment's effectiveness; or
(i) a method for identifying a biomarker for cancer treatment, comprising providing a plurality of non-human animals having one or more primary human cancer cells from a plurality of humans implanted in or on the liver of the plurality of animals; administering a cancer treatment to the plurality of animals; identifying animals among the plurality of animals in which the cancer treatment is ineffective; and identifying a biomarker that is common to the identified animals and is associated with (correlates with) the treatment's ineffectiveness; or
(j) a method for selecting cancer patients for a clinical trial, comprising providing a plurality of non-human animals having one or more primary human cancer cells from a plurality of humans implanted in or on the liver of the plurality of animals; administering a cancer treatment to the plurality of animals; identifying animals among the plurality of animals in which the cancer treatment is effective; identifying a biomarker that is common to the identified animals and is associated with (correlates with) the treatment's effectiveness; and including a patient in the clinical trial if the patient has the biomarker or excluding the patient from the clinical trial if the patient lacks the biomarker; or
(k) for selecting cancer patients for a clinical trial, comprising providing a plurality of non-human animals having one or more primary human cancer cells from a plurality of humans implanted in or on the liver of the plurality of animals; administering a cancer treatment to the plurality of animals; identifying animals among the plurality of animals in which the cancer treatment is ineffective; identifying a biomarker that is common to the identified animals and is associated with (correlates with) the treatment's ineffectiveness; and including a patient in the clinical trial if the patient lacks the biomarker or excluding the patient from the clinical trial if the patient has the biomarker.

18-51. (canceled)

52. An animal model comprising a non-human animal having one or more primary cancer cells implanted in or on the liver of said animal, wherein the primary cancer cells are from a species different from that of the animal.

53. The animal model of claim 52, wherein the primary cancer cells are from a non-human species.
